(19) United States
(12) Patent Application Publication
(10) Pub. No.: US 2025/0197932 A1
(43) Pub. Date: Jun. 19, 2025

(54) DISEASE SUBTYPE CLASSIFICATION USING GENOMIC FEATURES AND CLUSTERING

(71) Applicant: Foundation Medicine, Inc., Cambridge, MA (US)

(72) Inventors: Zoe R. Fleischmann, Jamaica Plain, MA (US); Brennan Decker, Winchester, MA (US); Ethan S. Sokol, Somerville, MA (US)

(73) Assignee: Foundation Medicine, Inc., Cambridge, MA (US)

(21) Appl. No.: 18/979,256

(22) Filed: Dec. 12, 2024

Related U.S. Application Data
(60) Provisional application No. 63/609,835, filed on Dec. 13, 2023.

(51) Int. Cl. C12Q 1/6874; C12Q 1/6855; G16B 20/10; G16B 20/20; G16B 40/30; G16H 15/00; G16H 50/50

(52) U.S. Cl. CPC C12Q 1/6874; C12Q 1/6855; G16B 20/10; G16B 20/20; G16B 40/30; G16H 15/00; G16H 50/50; C12Q 2600/112

(57) ABSTRACT

Techniques for performing prognostic classifications using unsupervised clustering are described. An example method includes determining features of a sample from a subject. The features, for instance, include an MMRD probability score of the sample and/or a copy number state of at least one genetic loci based on nucleic acid molecules of the sample. Input data is generated indicating the features. The example method further includes determining that the input data corresponds to at least one cluster in the clustering model and determining a prognostic classification of the subject based on the at least one cluster.

### CROSS-REFERENCE TO RELATED APPLICATION

This application claims priority to U.S. Provisional Application No. 63/609,835, which was filed on Dec. 13, 2023 and is incorporated by reference herein in its entirety.

### BACKGROUND

Biologically distinct subtypes of diseases frequently have widely divergent prognostic outlooks. For example, patients with different subtypes endometrial cancer may have vastly different mortality, morbidity, and appropriate treatment options. Clinical providers can determine the prognostic classification of a particular patient using a combination of genetic analysis, histological analysis, and other diagnostic testing. However, it may be difficult, costly, and time-consuming to determine the prognostic classification and the most accurate subtype classification is likely to require complex weighting of available data points. Further, with currently available techniques, despite this time and effort, prognostic classifications can be inaccurate.

### BRIEF DESCRIPTION OF THE DRA WINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

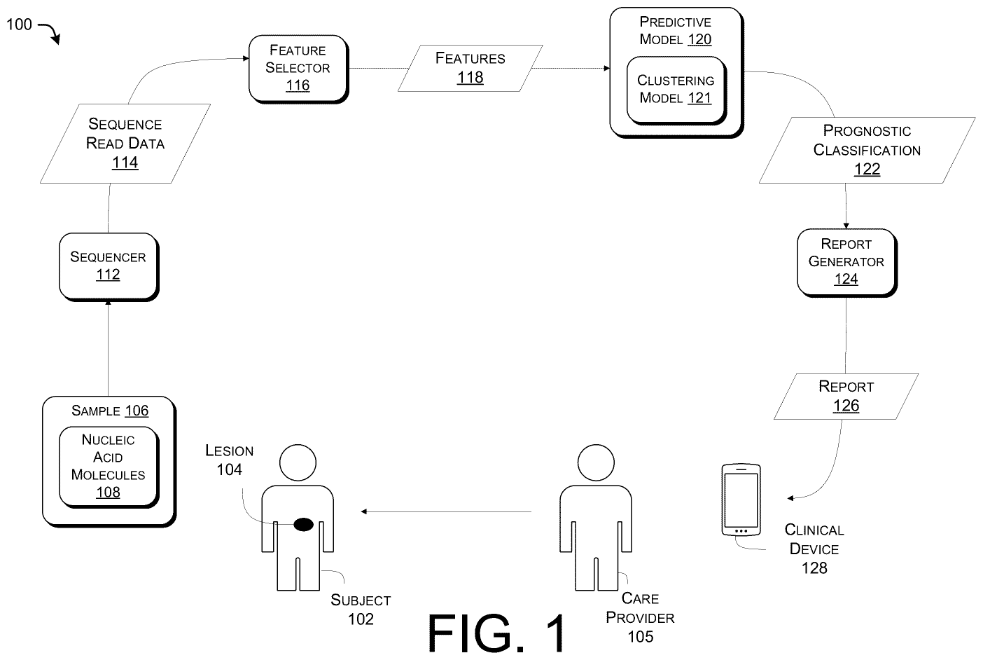

FIG. 1 illustrates an example environment for determining a prognostic classification of a subject.

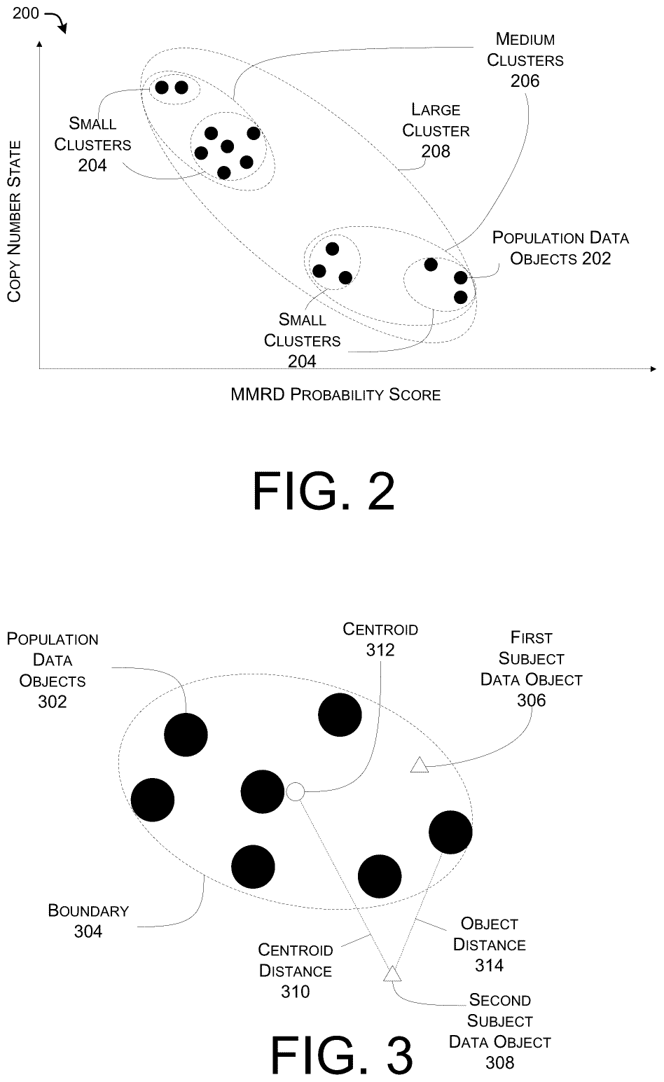

FIG. 2 illustrates an example of hierarchical clustering of data objects representing population features.

FIG. 3 illustrates an example of comparing subject data objects to a previously defined cluster for the purpose of prognostic classification.

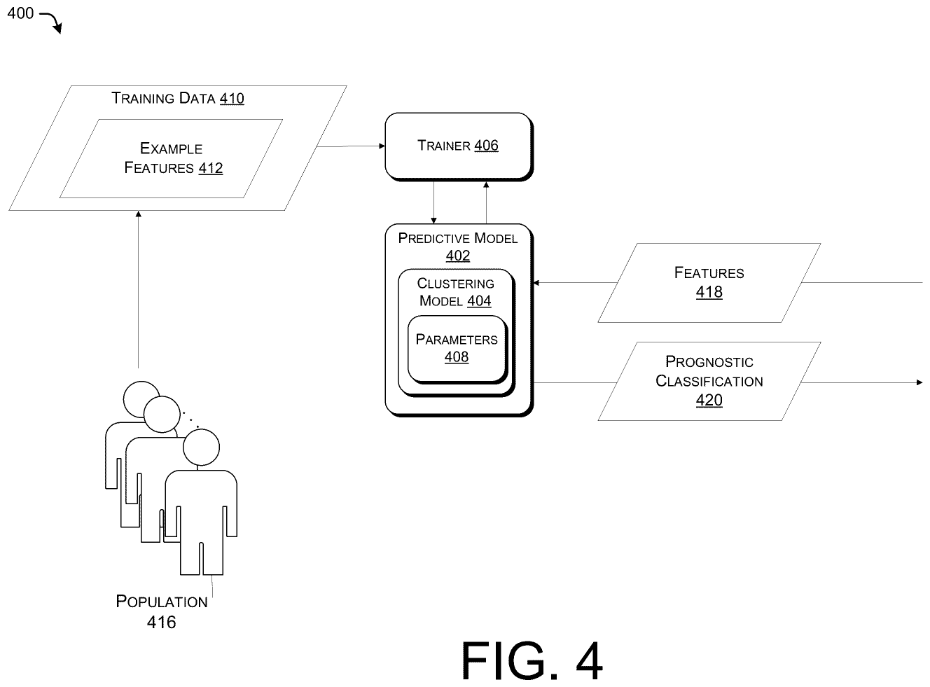

FIG. 4 illustrates an example environment for training and utilizing a predictive model to determine prognostic classifications of one or more diseases.

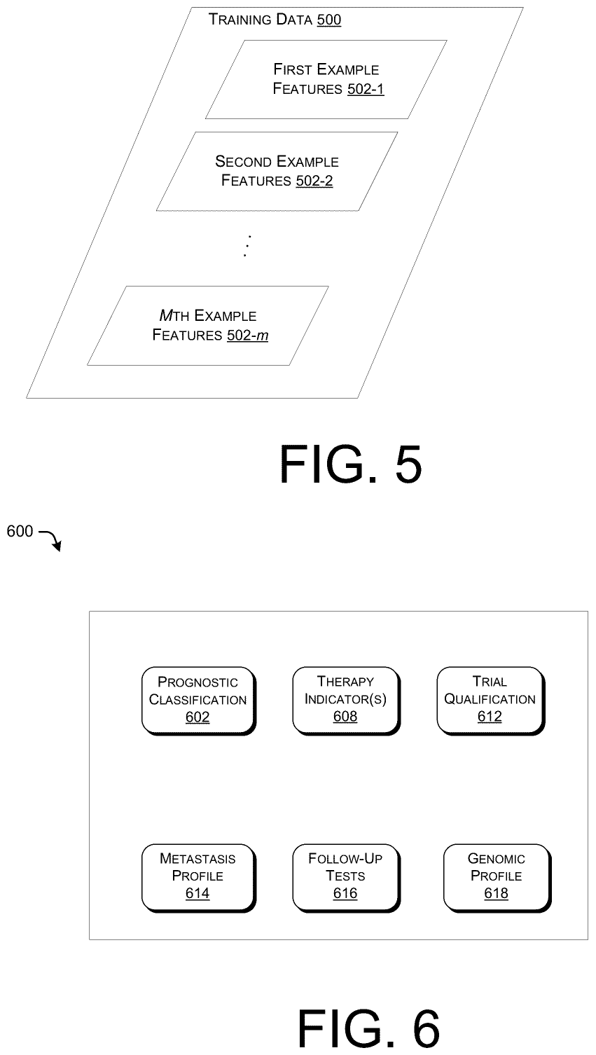

FIG. 5 illustrates an example of training data utilized to train one or more machine learning models.

FIG. 6 illustrates an example report summarizing predicted categories of a cancer of a subject.

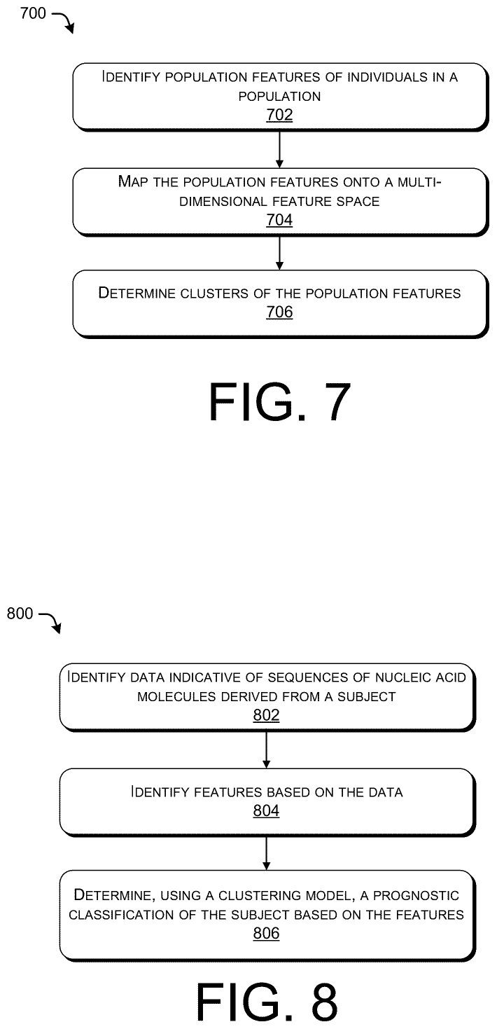

FIG. 7 illustrates an example process for clustering population features for the purposes of prognostic classification.

FIG. 8 illustrates an example process for performing prognostic classifications using clustering.

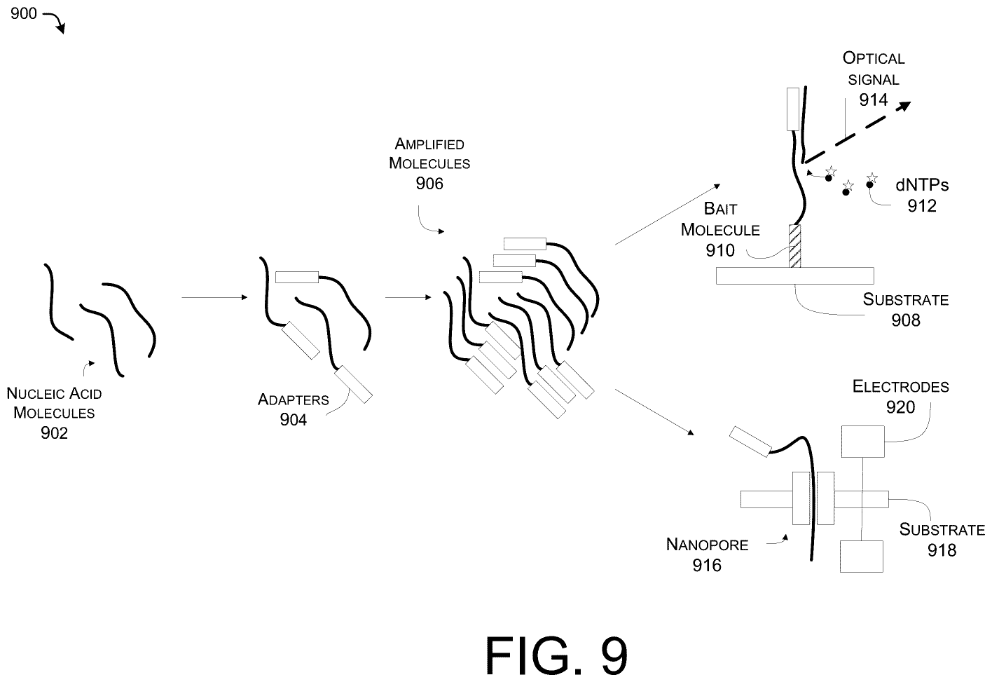

FIG. 9 illustrates an example environment for sequencing various nucleic acid molecules.

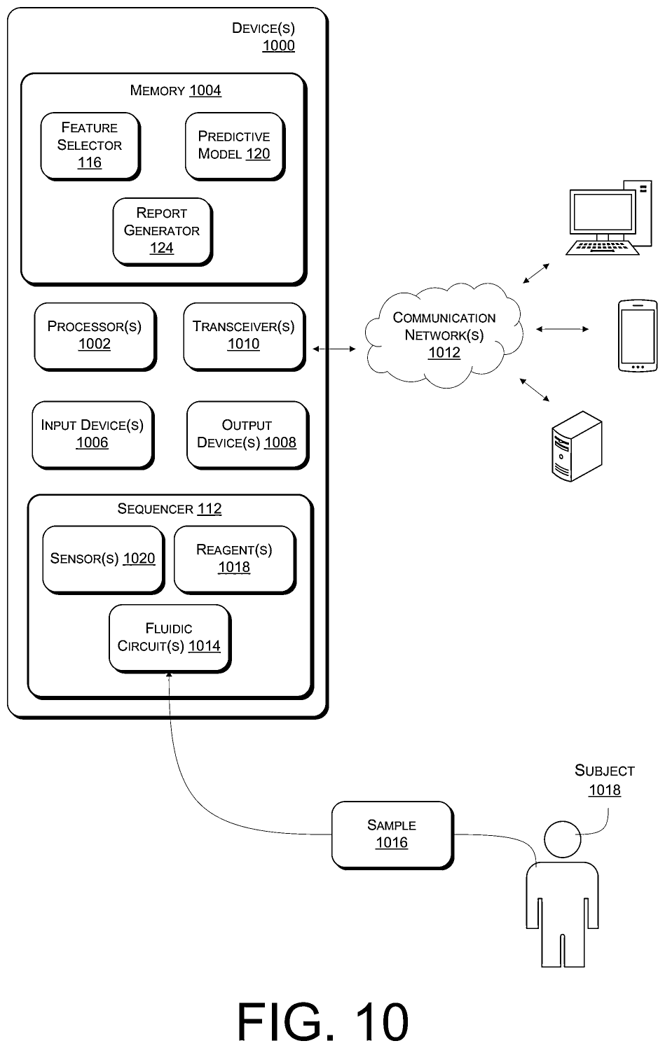

FIG. 10 illustrates one or more devices configured to perform various operations described herein.

### DETAILED DESCRIPTION

Various implementations of the present disclosure relate to techniques for classifying diseases and disease subtypes using clustering. For instance, techniques described herein can be used to accurately identify cancer subtypes of patients. In various examples, various features of a subject are obtained. In some cases, DNA and/or RNA in a sample obtained from the subject are sequenced. Genomic features are derived based on the sequenced DNA and/or RNA, in various examples. In various implementations, the genomic features are identified in a multi-dimensional feature space. The genomic features of the subject are compared to clusters that are also identified within the multi-dimensional feature space. In various cases, the genomic features are determined to be part of one of the clusters. The disease of the subject can therefore be classified based on the cluster in which the subject's genomic features belong.

In various examples, the clusters are established based on genomic features associated with a population of individuals that omit the subject. The genomic features of the population are mapped to the feature space, and the genomic features are grouped using a clustering technique (e.g., hierarchical clustering). In various cases, common prognostic features of the individuals whose genomic features are included in each cluster can be determined. The clusters may be labeled, or otherwise associated with, their corresponding prognostic features. Collectively, the prognostic features associated with a cluster may form a prognostic classification associated with that cluster. Accordingly, when the genomic features of the subject are determined to be part of a particular cluster, the subject may be determined to have the prognostic classification associated with the particular cluster.

Implementations of the present disclosure provide significant improvements to the technical field of cancer diagnosis and treatment. Previously, the prognostic classification of a tumor was dependent on genetic analysis and histological and/or immunohistological studies performed manually by a pathologist. The process of fixation, staining, and analysis in order to perform the histological and/or immunohistological studies could take days or weeks, which could lead to significant delays in diagnosis and prognostic classification. For certain pathologies, such as highly aggressive cancers, the delays in diagnosis and prognostic classification could lead to delays in treatment, which could cause significant harm to patients. Beyond this described delay, accuracy of the resultant diagnosis and prognostic classification could not be guaranteed. For patients with an inaccurate diagnosis and prognostic classification, additional significant harm ensues. Various implementations of the present disclosure relate to predictive models that are able to determine a prognostic classification with a high level of accuracy. Further, the predictive model may determine the prognostic classification in a relatively short amount of time, particularly when compared with histological studies.

Various analyses described herein cannot be performed in the human mind, or by pen and paper. In various implementations described herein, it would be impossible for a human to manually identify pertinent features of a genome and/or transcriptome of a subject. For instance, a human could not manually determine a mismatch repair deficiency (MMRD) probability score, a copy number state, a presence of a pathogenic variant, a fraction unstable score, a mutation signature; a tumor mutational burden (TMB) score, or the like, from sequence read data representing an entire genome of a subject. Further, it would be impossible for a human to manually predict a prognostic classification of the subject based on various pertinent features described herein. Implementations described herein are deeply rooted in computer technology, and are not mental processes that can be incidentally performed on a computer.

### Example Definitions

As used herein, the terms “deoxyribonucleic acid,” “DNA,” “DNA molecule.” and their equivalents, may refer to a polymer of nucleotides (also referred to as “nucleobases”) containing deoxyribose. The nucleotides in DNA include cytosine (C), guanine (G), adenine (A), and thymine (T). Each DNA nucleotide includes a deoxyribose and a phosphate group. An example single-stranded DNA (ssDNA) molecule includes a chain of covalently bonded DNA nucleotides. In the example ssDNA molecule, the phosphate group of the mth nucleotide is covalently bonded to the deoxyribose of the (m−1)th nucleotide, wherein m is a positive integer greater than 2 and less than or equal to the number of DNA nucleotides in the chain. In various examples, DNA is double-stranded and includes two ssDNA molecules that are complementary to one another and coiled around each other in a double helix form. The nucleotides of one ssDNA molecule are hydrogen bonded to the nucleotides of the other ssDNA molecule. In particular, the pyrimidines (A and T) hydrogen bond to each other, and the purines (C and G) hydrogen bond to each other.

As used herein, the terms “ribonucleic acid.” “RNA.” “RNA molecule,” and their equivalents, may refer to a polymer of nucleotides containing ribose. The nucleotides in RNA include cytosine (C), guanine (G), adenine (A), and uracil (U). Each RNA nucleotide includes a ribose and a phosphate group. In an example RNA molecule, the phosphate group of the nth nucleotide is covalently bonded to the ribose of the (n−1)th nucleotide, wherein n is a positive integer greater than 2 and less than or equal to the number of RNA nucleotides in the chain. Messenger RNA (mRNA) is a type of RNA molecule that is synthesized (or “transcribed”) by RNA polymerase (an enzyme) to be complementary to a gene encoded in a DNA sequence, and is also used by a ribosome to synthesize a polypeptide or protein. An mRNA is therefore an example of a “coding RNA.” In various cases, intron sequences are removed from an mRNA via a process known as “RNA splicing.” MicroRNA (“miRNA”) are single-stranded RNA molecules that perform post-transcriptional gene expression regulation. For instance, a miRNA may bind to a complementary mRNA molecule, thereby cleaving, destabilizing, or otherwise preventing the mRNA molecule from being translated into a polypeptide or protein by a ribosome. In various examples, a miRNA has a length in a range of 21 to 23 RNA nucleotides. As used herein, the terms “non-coding RNA” may refer to a type of RNA that is not translated into a protein. Examples of non-coding RNA include miRNA, transfer RNA (tRNA), and ribosomal RNA (rRNA). The term “functional RNA,” and its equivalents, may refer to any RNA molecule that impacts a biological process. For instance, functional RNA may include mRNA, miRNA, tRNA, rRNA, and the like.

As used herein, the term “base,” and its equivalents, may refer to a monomer of a polymer. For example, a base of DNA or RNA is a nucleotide.

As used herein, the term “base pair,” and its equivalents, may refer to a pair of complementary DNA nucleotides, which are hydrogen-bonded to one another in a double-stranded DNA molecule. For example, a base pair includes a first base in a first ssDNA and a second base in a second ssDNA, wherein the first and second bases are complementary and hydrogen-bonded to one another.

As used herein, the terms “nucleotide,” “nucleobase,” “nucleic acid,” “nucleic acid molecule,” and their equivalents, may refer to an organic molecule that includes a nitrogenous base, a sugar, and a phosphate group. In various cases, a nucleotide is a monomer of DNA or RNA. A nucleotide, for instance, is a chemical structure.

As used herein, the terms “3′ end,” “3-prime end.” and their equivalents, may refer to a terminus of a single-stranded nucleotide polymer that includes a base whose third carbon in its deoxyribose or ribose is bound to a hydroxyl group while being unbound to another base.

As used herein, the terms “5′ end.” “5-prime end.” and their equivalents, may refer to a terminus of a single-stranded nucleotide polymer that includes a base whose fifth carbon in its deoxyribose or ribose ring is unbound to another base. In some cases, the fifth carbon is bound to a phosphate group.

As used herein, the “length” of a polymer refers to a number of covalently bonded monomers that are included in the polymer. For instance, the length of a DNA molecule may be the number of covalently bonded nucleotides in at least one strand of the DNA molecule and/or the number of base pairs in the DNA molecule. In various examples, the length of an RNA molecule may be the number of covalently bonded nucleotides in the RNA molecule.

As used herein, the term “gene,” and its equivalents, refers to a sequence of DNA nucleotides that is transcribed into a functional RNA. The functional RNA, for instance, is RNA that is translated into a polypeptide or protein (e.g., mRNA) or that has some other biological function (e.g., miRNA, tRNA, etc.). A gene is “expressed” when it is used as a template to generate a functional RNA. A subject, for instance, has numerous genes contained in the subject's genome. A gene may include both introns and exons. As used herein, the term “intron,” and its equivalents, may refer to a subset of DNA nucleotides in a gene that is not used to code for any functional RNA that is expressed by the organism. As used herein, the term “exon,” and its equivalents, may refer to a subset of DNA nucleotides in a gene that is used to code for a functional RNA. For instance, an exon may encode a polypeptide or protein that is expressed by the organism. In various examples, a gene can be represented in data (e.g., as data representative of the sequence of DNA nucleotides in the gene) or as a chemical structure (e.g., as the sequence of DNA nucleotides itself).

As used herein, the term “genome,” and its equivalents, refers to the aggregate of genes of a subject. In various cases, a genome represents the sequences of several linear DNA molecules that are present in a subject's chromosomes. A “reference genome” refers to an aggregation of genes of one or more reference subjects. In various cases, a genome is represented in data.

As used herein, the terms “pangenome,” “pan-genome,” “supragenome,” and their equivalents, refers to an aggregate set of genes from multiple subgroups (e.g., strains) within a population (e.g., a clade) of subjects. A pangenome, for example, indicates genes that are present in all subjects within the population, as well as genes that are present in some of the subjects of the population. A pangenome is represented in data, for instance.

As used herein, the term “transcriptome,” and its equivalents, refers to the aggregate of RNA sequences of a subject. In some cases, a transcriptome is limited to mRNA sequences. In various examples, a transcriptome is represented in data.

As used herein, the term “genomic DNA,” “gDNA,” “chromosomal DNA,” and their equivalents, may refer to DNA molecules that are obtained from a chromosome and/or nucleus of a cell.

As used herein, the terms “DNA fragment,” “fragment,” and their equivalents, may refer to DNA molecules that are excised and/or broken off from a larger DNA molecule.

As used herein, the terms “cell-free DNA,” “cfDNA,” and their equivalents, may refer to DNA fragments that are non-encapsulated and obtained outside of cells within a sample (e.g., a liquid biopsy sample).

As used herein, the terms “circulating tumor DNA,” “ctDNA,” and their equivalents, may refer to a cfDNA molecule that originates from a cancer cell.

As used herein, the term “promoter,” and its equivalents, may refer to a portion of a DNA molecule that binds one or more proteins in order to initiate transcription of a gene. For example, the promotor is located “upstream” of the gene. For example, the promotor is located between 5′ end of the DNA molecule and the gene. A promotor may include one or more binding sites for RNA polymerase, and/or one or more transcription factor binding sites. In some examples, a promotor includes one or more CpG islands. A promoter, for instance, includes a transcription start site.

As used herein, the terms “CpG island,” “CGI,” “CpG site,” and their equivalents, may refer to a continuous portion of a DNA molecule whose sequence includes greater than a threshold amount (e.g., greater than 50%) of G-C base pairs.

As used herein, the term “enhancer,” and its equivalents, may refer to a portion of a DNA molecule that binds one or more proteins in order to increase the chance that a gene will be transcribed. For instance, an enhancer includes one or more transcription factor binding sites. In various cases, an enhancer includes one or more CpG islands.

As used herein, the term “cancer,” and its equivalents, may refer to a condition of a subject in which particular cells (referred to as “cancer cells”) divide uncontrollably in the subject's body. In some cases, a cancer is characterized by a location or tissue type from which the cancer cells originated. In some examples, a cancer is characterized by a location or tissue type in which the cancer cells are located.

As used herein, the terms “tumor,” “neoplasm,” and their equivalents, may refer to a mass of tissue including cancer cells.

As used herein, the terms “tissue of origin.” “tissue origin,” and their equivalents, refers to a differentiated type of tissue from which cancer cells in the body of a subject began dividing uncontrollably in the subject's body.

As used herein, the terms “liquid biopsy.” “fluid biopsy.” and their equivalents, may refer to a process of obtaining a fluid sample from a subject's body. The sample, for instance, can be referred to as a “liquid biopsy sample.” Examples of fluids that are sampled from the body include blood, plasma, cerebrospinal fluid, sputum, stool, urine, lymphatic fluid, and saliva.

As used herein, the term “tissue biopsy.” and its equivalents, may refer to a process of obtaining a sample of cells from a subject's body. A tissue biopsy, in various cases, is performed by cutting a mass of cells from the subject's body. For instance, a tissue biopsy is a procedure performed by a surgeon, interventional radiologist, interventional cardiologist, or other specialized clinician. The term “tissue” or “tissue biopsy sample” can be used to refer to the sample of cells obtained using a tissue biopsy.

As used herein, the term “subject.” and its equivalents, may refer to a human or non-human animal. A subject that is receiving care from at least one care provider may be referred to as a “patient.”

As used herein, the terms “machine learning.” “ML.” “computer learning.” “artificial intelligence,” and their equivalents, may refer to the use of at least one computing device to learn patterns in training data. The process of learning these patterns may be referred to as “training.” In particular cases, one or more computing devices may perform machine learning by executing a machine learning model. As used herein, the terms “machine learning model,” “ML model,” and their equivalents, may refer to data encoding instructions that, when executed by at least one computing device, causes the at least one computing device to learn patterns in training data by optimizing one or more metrics, values, or other types of parameters. After training, an ML model, when executed by at least one computing device, causes the at least one computing device to utilize the optimized parameters in order to perform one or more tasks.

As used herein, the term “variant.” and its equivalents, may refer to a difference between a subject genetic sequence and a reference sequence. For instance, a variant may correspond to a difference between one or more nucleotides in a genome of a subject and one or more corresponding nucleotides in at least one reference genome or pangenome. A variant may be characterized by its identity (e.g., what nucleotides are different), its position (e.g., where are the nucleotides located in the genome, what chromosome contains the nucleotides, what gene contains the nucleotides, etc.), its length (e.g., how many nucleotides are different from the reference sequence), its type (e.g., substitution, insertion, deletion, copy number alternation, rearrangement of fusion, etc.), and other features that indicates its significance and/or relevance. In some cases, a variant represents any apparent alteration in a sequence that has been read from a nucleic acid molecule with respect to the reference sequence, such as reads cleaved by restriction enzymes (RE). In various examples, a variant can be represented in data (e.g., by data characterizing the variant) or as a chemical structure (e.g., the nucleotides themselves). As used herein, the term “mutation,” and its equivalents, may refer to a change in a gene.

As used herein, the term “substitution,” and its equivalents, can refer to a nucleotide in a subject sequence that is different than an equivalent nucleotide (e.g., a nucleotide at the same position) in a reference sequence.

As used herein, the term “insertion,” and its equivalents, can refer to a nucleotide in a subject sequence that is added with respect to a reference sequence.

As used herein, the term “deletion,” and its equivalents, can refer to the removal of a nucleotide from a nucleotide sequence.

As used herein, the terms “copy number alternation.” “CNA.” “copy number variation,” “CNV.” and their equivalents, can refer to a portion of a reference sequence that is repeated.

As used herein, the terms “rearrangement of fusion,” “fusion rearrangement,” “translocation,” and their equivalents, can refer to a change in the relative position of one or more portions of a reference sequence, thereby generating a gene that was not present in the reference sequence.

As used herein, the term “sequencing.” and its equivalents, may refer to a process of identifying the order and identity of monomers in a polymer chain, such as the order and identity of nucleotides in a DNA or RNA molecule. The terms “whole genome sequencing.” “WGS.” and their equivalents, may refer to the process of sequencing an entire genome of a subject, including the introns and exons of the genes of the subject. The term “whole exome sequencing.” and its equivalents, may refer to the process of sequencing all exomes of a subject. The term “targeted sequencing.” and its equivalents, may refer to the process of sequencing a portion of the genome of a subject, such as sequencing a single gene of the subject. Various techniques can be utilized to sequence a DNA or RNA molecule, such as massively parallel sequencing (MPS), nanopore sequencing, direct sequencing, Sanger sequencing, or next-generation sequencing. In various cases, sequencing is performed on physical molecules (e.g., RNA or DNA) and is used to generate data.

As used herein, the terms “massive parallel sequencing,” “massively parallel sequencing,” “MPS,” and their equivalents, may refer to a technique for simultaneously performing multiple reactions that can be used to identify the order and identity of monomers in multiple polymer chains. In particular cases, massive parallel sequencing can be performed using sequencing-by-synthesis on clonally amplified DNA molecules that are located in spatially separated regions, which are individually monitored by sensors.

As used herein, the term “nanopore sequencing,” and its equivalents, may refer to a technique for identifying the order and identity of monomers in a polymer chain by transporting the polymer chain from a first space to a second space, wherein the first space and the second space are separated by a substrate, by directing the polymer chain through a small hole (known as a “nanopore”) embedded in the substrate, and monitoring a relative electrical signal (e.g., a voltage or current) between the first space and the second space.

As used herein, the term “sensor,” and its equivalents, may refer to a physical device or other apparatus that is configured to detect one or more detection signals.

As used herein, the term “detection signal,” and its equivalents, may refer to a physical signal that can be identified, characterized, or otherwise perceived by a sensor.

As used herein, the term “sequence read data,” and its equivalents, may refer to data that is indicative of an order and identity of monomers in a polymer, such as the order and identity of nucleotides in a DNA or RNA sequence. In various implementations, sequence read data is generated via a sequencing operation.

As used herein, the term “image,” and its equivalents, may refer to 2D or 3D array of data indicative of an array of pixels or voxels.

As used herein, the term “ligating,” and its equivalents, may refer to a process of joining two molecules together, for example, with a chemical bond.

As used herein, the term “adapter,” and its equivalents, may refer to an oligonucleotide that can be ligated to a target nucleic acid molecule. In various cases, an adapter prepares the target nucleic acid molecule for sequencing.

As used herein, the term “bait molecule,” and its equivalents, may refer to a nucleic acid molecule having a region that is complementary to a region of a target molecule (e.g., cfDNA). A bait molecule includes, for instance, a nucleic acid molecule that can hybridize to (i.e., is complementary to) a target molecule can be used to capture the target molecule. In some instances, the bait molecule is a capture oligonucleotide (or capture probe). In some instances, the bait molecule is suitable for solution phase hybridization to the target molecule. In some instances, the bait molecule is suitable for solid phase hybridization to the target molecule. In some instances, the bait molecule is suitable for both solution-phase and solid-phase hybridization to the target molecule. The design and construction of bait molecules is described in more detail in, e.g., International Patent Application Publication No. WO 2020/236941.

As used herein, the term “amplifying.” and its equivalents, may refer to a process of generating copies of a target molecule, such as a nucleic acid molecule.

As used herein, the term “hybridization.” and its equivalents, may refer to a process by which to complementary single-stranded nucleic acid molecules bind to one another, thereby forming a double-stranded nucleic acid molecule. In certain examples, the double-stranded nature of the nucleic acid molecule is maintained under stringent hybridization conditions. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C.

As used herein, the term “complementary.” and its equivalents, may refer to a state of two single-stranded nucleic acid molecules with respective sequences that cause the nucleic acid molecules to spontaneously hybridize to one another. One nucleic acid molecule, for instance, may have a sequence that causes each nucleic acid to hydrogen bond to a respective nucleic acid in the other nucleic acid molecule.

As used herein, the terms “therapy.” “treatment.” and their equivalents, may refer to a composition or process that can be used to remediate a health problem. Cancer therapies, for instance, include surgery, radiotherapy, chemotherapy, immunotherapy, cell-based therapies, and the like. Examples of cancer therapies include abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erlcada), asciminib hydrochloride (Scemblix), atczolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Belcodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx. Comctriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), clotuzumab (Empliciti), cnasidenib mesylate (Idhifa), encorafenib (Braftovi), enfortumab vedotin-cjfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), Lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximabcmkb (Margenza), midostaurin (Rydapt), mobocertinib succinate (Exkivity), mogamulizumab-kpkc (Poteligco), moxctumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyrc), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecanhziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), and combinations thereof. Examples of cancer therapies also include targeted antibody-based therapies (antibody-drug conjugates, antibody-radioisotope conjugates, and targeted immune cell therapies (e.g., immune effector cells genetically modified to express a chimeric antigen receptor (CAR).

As used herein, the term “treatment-responsive.” and its equivalents, may refer to a type of cancer cells that can be substantially killed using a predetermined type of therapy. For example, cancer cells of a subject may be responsive to a particular treatment if, after the subject is administered the treatment, the cancer cells are diminished by a particular progression level (e.g., radiographic progression level, marker-based progression level, such as prostate-specific antigen (PSA) progression, etc.). Accordingly, the responsiveness of the cells to the type of therapy may indicate the effectiveness of that therapy.

As used herein, the term “treatment-resistant,” and its equivalents, may refer to a type of cancer that cannot be substantially killed using a predetermined type of therapy.

As used herein, the term “metastasis profile,” and its equivalents, may refer to a propensity of a type of cancer to metastasize into one or more differentiated tumor types besides the cancer's tissue origin. In some implementations, the metastasis profile can further indicate the type of tissue in which the cancer can or is likely to metastasize.

As used herein, the term “clinical trial,” and its equivalents, may refer to a research study used to evaluate a hypothesis based on participation by one or more subjects. In various examples, a clinical trial can be used to assess the efficacy and/or safety of a proposed therapy. A clinical trial may be performed in furtherance of approval of a treatment by a regulatory authority (e.g., the United States Food & Drug Administration (FDA)).

As used herein, the terms “mismatch repair gene.” “MMR gene.” and their equivalents, may refer to a gene associated with DNA mismatch repair (MMR). MMR is a means by which cells can recognize and/or repair erroneous insertion, deletion, mis-incorporation, or other mutations that arise during DNA replication and recombination. In some cases, MMR enables cells to repair damage to DNA. Examples of MMR genes include, for instance, MSH2, MSH6, PMS2, or MLH1.

As used herein, the terms “mismatch repair deficiency probability score.” “MMRD probability score.” and their equivalents, may refer to a likelihood that one or more MMR pathways (e.g., in cells of a subject and/or a sample) are ineffective at performing mismatch repair.

As used herein, the term “clustering model,” and its equivalents, may refer to a computing model that is configured to group multiple data objects by similarity. In various examples, a clustering model may operate as an unsupervised ML model that identifies patterns within a dataset.

As used herein, the term “hierarchical clustering.” and its equivalents, may refer to techniques for grouping data objects by generating a hierarchy of different object groups. As used herein, the term “agglomerative hierarchical clustering.” and its equivalents, may refer to techniques for grouping data objects by merging clusters of the data objects in accordance with a distance metric. For instance, each data object may initially be assigned an individual cluster, and those clusters may be merged based on the Euclidian distance between the data objects and/or clusters. As used herein, the term “divisive hierarchical clustering.” and its equivalents, may refer to techniques for grouping data objects by separating clusters of the data objects in accordance with a distance metric. For instance, all data objects may be initially grouped in a single cluster that is separated into multiple clusters based on the Euclidian distance between the data objects and/or clusters.

As used herein, the terms “multi-dimensional scaling,” “MDS,” and their equivalents, may refer to a technique by which data objects are represented in a feature space, wherein the distances between the data objects in the feature space are representative of similarities between the data objects.

As used herein, the terms “pathology,” “disease,” “medical condition,” and their equivalents, may refer to an abnormal condition of a subject that adversely impacts the function and/or structure of at least a portion of the subject. Cancers are examples of diseases. Diseases may include, for instance, infectious diseases (e.g., diseases caused by viruses, bacteria, or other infectious agents), deficiency diseases (e.g., malnutrition or other disease associated with a lack of one or more nutrients), hereditary diseases (e.g., diseases triggered by genomic features of the subject), physiological diseases (e.g., diabetes and other diseases in which a portion of the subject's body malfunctions), or combinations thereof.

As used herein, the terms “pathology subtype,” “disease subtype,” and their equivalents, may refer to one or more distinct clinical features, prognostic features, comorbidities, progression, or any combination thereof, that are associated with some, but not all, subjects having the same disease. For example, a single disease may be associated with different disease subtypes.

As used herein, the term “pathogenic variant,” and its equivalents, may refer to a variant that is associated with at least one disease. In some cases, the pathogenic variant is a characteristic of at least one disease. In some examples, the pathogenic variant is associated with an increased likelihood that at least one disease is present.

As used herein, the term “fraction unstable score,” and its equivalents, may refer to a metric (e.g., a number) that is indicative of mutations in microsatellites and/or other portions of the genome that are prone to mutations.

As used herein, the terms “tumor mutational burden” score, “TMB score,” and their equivalents, may refer to a metric indicative of the number of mutations carried by tumor cells. For instance, a TMB score may be a number of somatic mutations in the genome of cells in a tumor and/or the number of somatic mutations per area of the genome of the cells in the tumor. In some cases, the TMB score is a measure of the number of somatic mutations per megabase (Mb) of DNA sequenced. In some examples, germline (inherited) variants are excluded when determining TMB, given that the immune system has a higher likelihood of recognize these variants as self. In various cases, driver mutations are excluded from the TMB score calculation.

As used herein, the term “tumor purity;” and its equivalents, may refer to a metric indicative of the number of tumor cells (e.g., with respect to the total number of cells and/or the number of non-tumor, “normal” cells) in a sample. Techniques for calculating tumor purity are described, for instance, in PCT/US22/79043. In some examples, multiple (e.g., three) statistical moments are calculated based on sequencing depth signals of a sample that includes a mixture of tumor cells and non-tumor cells. The statistical moments may refer to parameters that characterize the shape of a distribution. The tumor purity may be calculated by solving a set of nonlinear equations generated using the statistical moments. In some instances, the nonlinear equations represent equating the calculated statistical moments with theoretical moments. In some examples, tumor purity can be calculated based on depth of coverage and minor allele frequency (MAF) information of the sample.

As used herein, the term “copy number state.” and its equivalents, may refer to a metric that is indicative of a number of copies of one or more loci in the genome of a subject and/or a sample obtained from the subject. For example, the copy number state may refer to the number of copies of a predetermined sequence at a given locus in the genome of the subject and/or the sample.

As used herein, the term “microsatellite instability score,” “MSI score,” and its equivalents, may refer to an amount of instability in one or more microsatellites. For example, an MSI score can be represented as a fraction (i.e., an “MSI fraction”) of instability in the one or more microsatellites. In certain examples. “microsatellite” refers to a repetitive nucleic acid having repeat units of less than about 10 base pairs or nucleotides in length. In certain examples, a microsatellite refers to a tract of tandemly repeated (i.e. adjacent) DNA motifs ranging from one to six or up to ten nucleotides, with each motif repeated 5 to 50 repeated times.

As used herein, the term “mutational profile,” and its equivalents, may refer to a dataset characterizing multiple variants associated with a predetermined disease, such as a cancer type or subtype. In some cases, a similarity between variants observed in a sample and a predetermined mutational profile may be indicative of whether the subject from which the sample was obtained has the predetermined disease associated with the predetermined mutational profile. Examples of mutational profiles are described, for instance, in Goncearenco et al., Nucleic Acids Res. 2017; 45 (W1): W514-W522. In some cases, a model (e.g., an autoencoder model) can parse a predetermined disease of a subject based on the data indicative of the mutational profile, or one or more mutational profiles.

As used herein, the term “aneuploidy event.” and its equivalents, may refer to an abnormal number of chromosomes in a cell. For human subjects, an aneuploidy event occurs if there are greater or fewer than 46 chromosomes. In some cases, a chromosome may be missing from a cell in a sample. In some examples, an additional chromosome is present in the cell of the sample.

As used herein, the term “prognostic classification,” and its equivalents, may refer to one or more characteristics (or expected characteristics) of an individual with a disease regarding the individual's symptoms, quality of life, comorbidities, disease development, disease duration, likelihood of survival (e.g., at a particular point in time), other prognostic characteristics, or any combination thereof. In some cases, a disease is associated with a finite number of prognostic classifications, such that

As used herein, the term “survivability,” and its equivalents, may refer to a likelihood that an individual will survive up to a predetermined point in time. For example, a 30-day survivability may refer to an individual's likelihood that they will survive the next 30 days, a five-year survivability may refer to an individual's likelihood that they will survive the next five years, and the like.

As used herein, the term “symptom,” and its equivalents, may refer to a feature that is indicative, or caused by, an underlying disease.

As used herein, the term “effective therapy.” and its equivalents, may refer to a therapy that is capable of treating a disease or symptom of the disease.

As used herein, the term “dose.” “dosage,” and their equivalents, may refer to an amount, frequency, schedule, formulation, or any combination thereof, of a therapy administered to a subject.

As used herein, the terms “data object.” “object.” and their equivalents, refers to a software structure that includes, or otherwise defines, multiple metrics, variables, values, data, or any combination thereof. For example, a single patient may be associated with multiple metrics (e.g., Boolean values, strings, number values, etc.) that are indicative of genomic features of a sample obtained from the patient, such as copy number state, MMRD probability score, the presence of a particular variant, and so on. A data object that includes each of these metrics can be defined for the patient. In some cases, the data object can be represented as a table, vector, list, or some other format.

### Description of Example Implementations

Various implementations of the present disclosure will now be described with reference to the accompanying Figures.

FIG. 1 illustrates an example environment 100 for determining a prognostic classification of a subject 102. In various implementations, the subject 102 has a disease or a suspected disease. The subject 102, for instance, may present to a clinical environment with a lesion 104. In various cases, the lesion 104 may be a tumor that includes cancer cells. According to various examples, the subject 102 has one or more types of cancer, such as adrenal cancer, bladder cancer, blood cancer, bone cancer, brain cancer, breast cancer, carcinoma, cervical cancer, colon cancer, colorectal cancer, corpus uterine cancer, car, nose and throat (ENT) cancer, endometrial cancer, esophageal cancer, gastrointestinal cancer, head and neck cancer, Hodgkin's disease, intestinal cancer, kidney cancer, larynx cancer, leukemia, liver cancer, lymph node cancer, lymphoma, lung cancer, melanoma, mesothelioma, myeloma, nasopharynx cancer, a neuroblastoma, non-Hodgkin's lymphoma, oral cancer, ovarian cancer, pancreatic cancer, penile cancer, pharynx cancer, prostate cancer, rectal cancer, sarcoma, seminoma, skin cancer, stomach cancer, a teratoma, testicular cancer, thyroid cancer, uterine cancer, vaginal cancer, a vascular tumor, or combinations or metastases thereof.

In some embodiments, the subject 102 has a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypercosinophilia, chronic cosinophilic leukemia, neuroendocrine cancers, or a carcinoid tumor.

In some embodiments, the subject 102 has acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), a non-small cell lung cancer KRAS (+/−G12C), a non-small cell lung cancer TMB-H, a non-small cell lung cancer MET exon 14 skipping, a non-small cell lung cancer ERBB2 inframe indel, a non-small cell lung cancer EGFR exon 20 indel, a neurotrophic tyrosine receptor kinase (NTRK)-positive cancer, ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia. In particular examples, the subject 102 has endometrial cancer. For instance, the lesion 104 includes an endometrial tumor that is present in endometrial tissue of the subject 102.

In various cases, a care provider 105 (also referred to as a “healthcare provider”) is responsible for diagnosing and/or treating the subject 102. According to some implementations, the lesion 104 may be initially identified using a noninvasive technique. For example, the lesion 104 may be visualized using an imaging modality, such as ultrasound, x-ray, computed tomography (CT), magnetic resonance imaging (MRI), positron emission tomography (PET), single photon emission CT (SPECT), or any combination thereof. Using the noninvasive technique, the care provider 105 may identify the presence of the lesion 104 but may be unable to determine whether the lesion 104 is a cancerous tumor using noninvasive diagnostic methodologies. In some cases in which the lesion 104 is a tumor, the care provider 105 may be unable to identify whether the tumor is metastatic or benign, or may be unable to otherwise categorize the tumor.

In various implementations, the care provider 105 is unable to determine a prognostic classification of the lesion 104 (e.g., a tumor) using noninvasive techniques. As used herein, the term “prognostic classification,” and its equivalents, may refer to a characteristic of a subject presenting with a disease (e.g., cancer), wherein the characteristic is determinative of, or at least correlated with, an effectiveness of at least one therapy at treating the disease, an ineffectiveness of at least one therapy at treating the disease, a survivability (e.g., a likelihood that the subject will survive by a predetermined date or time), an expected quality of life, at least one predetermined symptom, at least one comorbidity, another factor relevant to the prognosis associated with the disease, or any combination thereof.

The care provider 105 could determine a prognostic classification of the lesion 104 by initiating a tissue biopsy on the subject 102. For instance, the care provider 105 could surgically remove a tissue sample from the lesion 104 and/or review the tissue sample using histochemistry and/or immunohistochemistry. However, attempting to classify the lesion 104 using these techniques has several drawbacks. First, the tissue sample may not be classifiable using conventional histological techniques, such as conventional immunohistochemical staining and review. Second, it is unlikely that the single care provider 105 would be trained to perform the tissue biopsy (which would be performed by a surgeon), to administer anesthesia to the subject 102 during the tissue biopsy (which would be performed by an anesthesiologist), and the analysis of the tissue biopsy (which would be performed by a trained pathologist), such that the classification would utilize multiple highly trained care providers. Even if the lesion 104 was classifiable by these means, the coordinated efforts of these care providers could delay classification of the lesion 104 and could cause significant expense to the subject 102. In various examples, the delay in classification could cause significant emotional hardship to the subject 102, who could be prevented from receiving an informed prognosis for weeks. Further, the delay in classification could delay a therapy of the lesion 104, which could cause lasting harm to the subject 102, particularly in cases in which the lesion 104 is representative of an aggressive form of cancer.

In various implementations of the present disclosure, a prognostic classification of the lesion 104 is determined without performing histochemistry and/or immunohistochemistry. For instance, a sample 106 is obtained from the subject 102. In some examples, the sample 106 includes a tissue biopsy sample. For instance, the sample 106 is obtained by removing cells from the lesion 104 and from the subject 102. In some cases, the tissue biopsy sample is surgically excised from the subject 102. In some cases, the sample includes a liquid biopsy sample. The liquid biopsy sample 106, for instance, includes blood, plasma, cerebrospinal fluid, sputum, stool, urine, lymphatic fluid, saliva, or some other fluid obtained from the body of the subject 102. In some cases, a blood sample is obtained intravenously from the subject 102. The liquid biopsy sample 106, according to various examples, is a plasma sample obtained from the blood of the subject 102. The liquid biopsy sample 106 can be obtained in a minimally invasive procedure, which could be performed by a medical technician rather than a surgeon.

The sample 106 includes nucleic acid molecules 108. According to some examples, the nucleic acid molecules 108 include genomic DNA (gDNA). For instance, the nucleic acid molecules 108 include chromosomal DNA that is located in, or extracted from, cells in the sample 106. According to some cases, the DNA is extracted from nuclei and the cells in the sample 106 using mechanical shearing and/or the introduction of a chemical (e.g., a detergent). The DNA may be subsequently isolated from proteins and other cellular materials. In some implementations, the nucleic acid molecules 108 indicate an entire genome of the subject 102 and/or the lesion 104. Thus, a genome of the subject 102 and/or the lesion 104 can be determined by sequencing the DNA in the nucleic acid molecules 108.

In some examples, the nucleic acid molecules 108 include RNA. In some implementations, the nucleic acid molecules 108 include messenger RNA (mRNA), microRNA, non-coding RNA, functional RNA, or any combination thereof. Various RNA in the nucleic acid molecules 108 may be indicative of proteins expressed in the cells of the subject 102 and/or the lesion 104.

In some cases, the sample 106 includes cell-free DNA (cfDNA). In examples in which the subject 102 has cancer (e.g., the lesion 104 is a cancerous tumor), the cfDNA, for instance, includes circulating tumor DNA (ctDNA) and/or non-ctDNA. In cases wherein the lesion 104 is a tumor, cancer cells within the lesion 104 will lyse and release the ctDNA into the bloodstream of the subject 102. These cancer cells, for example, include circulating tumor cells (CTCs). Further, other cells additionally release non-ctDNA into the bloodstream of the subject. In general, the cfDNA includes fragments with lengths that are in a range of 1 to 500, 3 to 500, or 100 to 500 bases long. For instance, the cfDNA includes fragments that are about 170 bases long and/or fragments that are about 340 bases long. For example, the cfDNA includes fragments that are 100 to 240 bases long and/or fragments that are 270 to 410 bases long.

In various cases, the sample 106 is transported to a location that is remote from the subject 102 for further processing. For example, the sample 106 is removed from the subject 102 in a clinical environment (e.g., a hospital) and is then transported to a remote laboratory for further testing and analysis.

A sequencer 112 is configured to generate sequence read data 114 indicating the sequences of the nucleic acid molecules 108. The sequencer 112, for instance, includes one or more devices that are configured to generate the sequence read data 114 by processing at least a portion of the sample 106. In some cases, the nucleic acid molecules 108 are extracted from the sample 106. The extraction can be performed by the sequencer 112, by another device, manually (e.g., by a laboratory technician), or any combination thereof. Any appropriate extraction method known to those of ordinary skill in the art can be utilized.

In various cases, the sequencer 112 is configured to perform one or more processes (e.g., chemical reactions) on the nucleic acid molecules 108 in order to prepare the nucleic acid molecules 108 for sequencing. For instance, the sequencer 112 may ligate adapters onto the nucleic acid molecules 108 and/or amplify the nucleic acid molecules 108, such that numerous copies of the ligated nucleic acid molecules 108 are available for sequencing. Examples of the adapters include, for example, amplification primers, flow cell adapter sequences, substrate adapter sequences, or sample index sequences. The nucleic acid molecules 108 (e.g., the ligated nucleic acid molecules 108) may be amplified by generating multiple copies of the nucleic acid molecules 108 using one or more techniques such as polymerase chain reaction (PCR), a non-PCR amplification technique, or an isothermal amplification technique.

The sequencer 112 may identify the length, position, and identity of the bases in the nucleic acid molecules 108 by sequencing the nucleic acid molecules 108 (e.g., the amplified and/or ligated nucleic acid molecules 108). In various implementations, the sequencer 112 utilizes first-generation sequencing (e.g., Sanger sequencing), second-generation sequencing (e.g., massive parallel sequencing), third-generation sequencing (e.g., nanopore sequencing), or a combination thereof. In some cases, the sequencer 112 is configured to sequence substantially all of the nucleotides of all of the nucleic acid molecules 108 fragments obtained from the sample 106. In some examples, the sequencer 112 is configured to perform targeted sequencing. For instance, the sequencer 112 may determine whether the nucleic acid molecules 108 fragments contain one or more predetermined sequences at one or more genomic locations.

In various cases, the sequencer 112 includes one or more sensors that are configured to detect physical signals (also referred to as “detection signals”) that are indicative of the nucleotide sequences of the nucleic acid molecules 108. The sequencer 112 may perform sequencing-by-synthesis. For example, the sequencer 112 may include one or more optical sensors configured to detect optical signals emitted from fluorescently tagged nucleotide triphosphates (NTPs) that are joined together in a synthesized DNA strand using the ligated nucleic acid molecules 108 as templates. The optical signals detected by the optical sensor(s), for instance, are indicative of the sequences of the nucleic acid molecules 108. The sequencer 112 may perform nanopore sequencing. In various cases, the sequencer 112 includes one or more electrical sensors configured to measure an electrical signal (e.g., an electrical current) across a substrate as the ligated nucleic acid molecules 108 are directed through a nanopore extending through the substrate. The electrical signal over time, in various cases, is indicative of the sequences of the nucleic acid molecules 108 in the sample 106. The sequencer 112, in various implementations, is configured to generate the sequence read data 114 as digital data based on the analog signals detected by the sensor(s). For instance, the sequencer 112 includes one or more analog to digital converters (ADCs). In various cases, the sequencer 112 includes at least one processor configured to generate the sequence read data 114.

In some implementations, the sequencer 112 performs RNA sequencing (RNA-seq) on the nucleic acid molecules 108. For example, the nucleic acid molecules 108 include RNA that is extracted from the sample 106. In some examples, the RNA in the nucleic acid molecules 108 is fragmented. In various implementations, complementary DNA (cDNA) is generated using reverse transcriptase, such that the cDNA includes sequences that are complementary to the RNA in the nucleic acid molecules 108 from the sample 106. The cDNA, according to various cases, can be sequenced using the DNA sequencing techniques described above. Accordingly, in some cases, the sequence read data 114 indicates sequences of RNA present in the sample 106, which may be indicative of the transcriptome of the subject 102 and/or the lesion 104.

In various cases, the sequencer 112 performs sequencing on a subset of the nucleic acid molecules 108. For instance, the sequencer 112 may perform targeted sequencing on one or more predetermined genes, such as any of the genes described herein. The sequencer 112, in some cases, may refrain from sequencing at least a portion of the nucleic acid molecules 108 that do not correspond to the subset.

A feature selector 116 identifies features 118 of the nucleic acid molecules 108 by analyzing the sequence read data 114. In various implementations, the feature selector 116 identifies, calculates, or otherwise determines the features 118 based on the sequences of the nucleic acid molecules 108 indicated in the sequence read data 114. One or more types of features are identified by the feature selector 116. In various implementations, the features 118 are genomic features (also referred to as “genomic features”). That is, the features 118 may be derived from the sequence read data 114.

In some cases, the features 118 include a mismatch repair deficiency (MMRD) probability score. In various cases, the MMRD probability score indicates a likelihood that one or more MMR pathways of cells in the sample 106 are ineffective at performing mismatch repair. In some implementations, the MMRD probability score is determined by determining genomic features by analyzing the sequence read data 114, inputting the genomic features into at least one trained machine learning model trained to generate the MMRD probability score based on previously analyzed data from a population omitting the subject 102. The genomic features relevant to the MMRD probability score include, for instance, a fraction unstable score, a composite COSMIC single-base substitution signature, a COSMIC indel signature, a copy number signature, a tumor mutational burden score, a blood-based tumor mutational burden score, a germline status for a mutation in one or more genes associated with DNA mismatch repair (MMR) (also referred to as “MMR genes”), a methylation status for the one or more MMR genes, a methylation status for one or more promoters associated with the one or more MMR genes, a methylation status of one or more enhancers associated with the one or more MMR genes, or any combination thereof. Examples of the MMR genes include, for instance, MSH2, MSH6, PMS2, or MLH1.

The features 118, in some examples, include a copy number state of one or more genetic loci indicated by the sequence read data 114. In various implementations, a number of copies of a predetermined sequence at a given locus in the genome of the subject 102 and/or the lesion 104 (also referred to as a “copy number” of the locus) is determined. The copy number state, in various implementations, may indicate copy numbers of one or more loci in the genome of the subject 102 and/or the lesion 104. For instance, the copy number state may indicate the presence and/or amount of copies of various sequences present in the genome of the subject 102 and/or the lesion 104, which may be due to copy number variation.

According to various examples, the sequence read data 114 may represent a genome of the subject 102 and/or the lesion 104. Various portions of the sequence read data 114 are aligned with at least one reference sequence (e.g., a reference genome). The aligned data is segmented using at least one segmentation technique (e.g., a circular binary segmentation (CBS) method, a maximum likelihood method, a hidden Markov chain method, a walking Markov method, a Bayesian methods, a long-range correlation method, a change point method, or any combination thereof), thereby generating non-overlapping segments of the sequence read data 114, wherein a sequence associated with a given segment is associated with the same copy number (e.g., a number of instances in which the sequence appears in the segment). Various genetic loci are binned, or otherwise sorted, with respect to the segments of the genome of the subject 102 and/or the lesion 104. The copy number state, for instance, is representative of the respective copy numbers associated with the genetic loci.

In some implementations, the features 118 include the presence or absence of a variant (e.g., a pathogenic variant) in one or more genes associated with classifying the lesion 104. In various cases, the genes include one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNFIA, HRAS, HSD3B1, ID3, IDH1, IDH2, IGFIR, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRB, PDK1, PIK3C2B, PIK3C2G, PIK3CA, PIK3CB, PIK3R1, PIM1, PMS2, POLD1, POLE, PPARG, PPP2RIA, PPP2R2A, PRDM1, PRKARIA, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, or ZNF703. In some cases, the genes include one or more of ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1B, IL-6, IL-6R, JAK1, JAK2, JAK3, KIT, KRAS, MEK, MET, MSI-H, mTOR, PARP, PD-1, PDGFR, PDGFRa, PDGFRB, PD-L1, PI3K8, PIGF, PTCH, RAF, RANKL, RET, ROS1, SLAMF7, VEGF, VEGFA, or VEGFB.

In particular examples in which the lesion 104 is an endometrial tumor, relevant genes may include TP53, PTEN, POLE, MKI67, FAT3, TAF1, ZFHX3, RPL22, SPTA1, FAM135B, CSMD3, GIGYF2, CSDE1, MLL4, ATR, CTNNB1, USH2A, LIMCH1, RRN3P2, FBXW7, CDH19, USP9X, COL11A1, BCOR, ARIDIA, ZNF770, ARID5B, SLC9A11, KRAS, PNN, INPP4A, CTCF, CHD4, AMY2B, RBMX, PPP2RIA, TNFAIP6, PIK3R1, SGK1, HOXA7, METTL14, HPD, MIR1277, CCND1, MECOM, NFE2L2, ERBB2, PMS2, MSH2, MSH6, MLH1, and ESR1. For instance, the features 118 may include the presence of one or more pathogenic variants in POLE, TP53, CTNNNB1, LICAM, PTEN, ERBB2, PMS2, MSH2, MSH6, MLH1, an estrogen receptor (ER) gene (e.g., ESR1, ESR2, etc.), a progesterone receptor (PR) gene (e.g., PGR).

In some cases, the features 118 are indicative of microsatellite instability (MSI). Microsatellites are highly polymorphic DNA-repeat regions. In certain examples. “microsatellite” refers to a repetitive nucleic acid having repeat units of less than about 10 base pairs or nucleotides in length. In certain examples, a microsatellite refers to a tract of tandemly repeated (i.e. adjacent) DNA motifs ranging from one to six or up to ten nucleotides, with each motif repeated 5 to 50 repeated times. During DNA replication, mutations (e.g., insertions or deletions) are more likely to be introduced at microsatellites than various other portions of the genome. In various cases, these mutations are corrected via MMR pathways. However, if the MMR pathways are impaired (e.g., the MMR genes of the hosting cell include variants that impede function), then the mutations at the microsatellites may be substantially retained. “Microsatellite instability” refers to genetic instability in the microsatellite regions. Cancer patients with microsatellite instability classified as being high (MSI-H or MSI-High) frequently exhibit an accumulation of somatic mutations in tumor cells that leads to a range of molecular and biological changes including high tumor mutational burden, increased expression of neoantigens and abundant tumor-infiltrating lymphocytes. Chang et al. “Microsatellite Instability: A Predictive Biomarker for Cancer Immunotherapy.” Appl Immunohistochem Mol Morphol, 26 (2): e15-e21 (2018). These changes have been linked to increased sensitivity to checkpoint inhibitor drugs, such as pembrolizumab, which is used to treat advanced melanoma, head and neck squamous cell carcinoma, non-small cell lung cancer (NSCLC), and classical Hodgkin lymphoma. According to various examples. “MSI score” refers to an amount of instability in one or more microsatellites. For example, an MSI score can be represented as a fraction (i.e., an “MSI fraction”) of instability in the one or more microsatellites. Other types of portions of DNA may be associated with a high likelihood of mutations. In some cases, the features 118 include a fraction unstable score, indicative of mutations in the microsatellites and other portions of the genome that are prone to mutations.

In various cases, an MSI score can be determined based on a predetermined set of repetitive loci (e.g., 2000 repetitive loci, each with a minimum of 5 repeat units of mono-, di-, and trinucleotides). By evaluating the sequence read data 114, the feature selector 116 may determine lengths of repetitive sequences corresponding to the loci. If an example locus among the loci corresponds to a predetermined repeat length, the locus is considered to be “unstable.” The MSI score, for instance, is determined by determining an amount of the unstable loci (e.g., a fraction of the unstable loci with respect to the total number of repetitive loci evaluated). In some cases, the MSI score is used to determine whether the subject 102 and/or lesion 104 is MSI-High (MSI-H). For example, MSI-H status may be applicable if the MSI score is greater than a threshold (e.g., 0.5%). Techniques for determining MSI scores are described, for instance, in Woodhouse et al., “Clinical and analytical validation of FoundationOne LiquidCDx, a novel 324-Gene cfDNA-based comprehensive genomic profiling assay for cancers of solid tumor origin,” PLOS ONE 15(9) (2020).

In some implementations, the features 118 include a mutation signature. In various cases, a mutational signature can represent an amount and/or identity of mutations (e.g., insertions, deletions, double-base substitutions, single-base substitutions, or any combination thereof) indicated in the nucleic acid molecules 108 from the subject 102. In some cases, the mutational signature indicates an amount (e.g., number or percentage) of individual classes of base substitutions present in the nucleic acid molecules 108. For instance, the classes include single-base substitutions including C>A, C>G, C>T, T>A, T>C, and T>G. A mutational signature can be derived by comparing the sequences indicated in the sequence read data 114 to at least one reference sequence, such as a reference genome. For example, the features 118 may include a Catalogue Of Somatic Mutations In Cancer (COSMIC) mutational signature, such as a COSMIC indel signature. In some cases, the features 118 include a single-base substitution signature.

In various examples, the features 118 include a tumor mutational burden (TMB) score. Tumor mutational burden (TMB) is a measure of the number of mutations carried by tumor cells. By comparing DNA sequences from a patient's healthy tissues and tumor cells, the number of acquired somatic mutations present in tumors, but not in normal tissues, may be determined. In some instances, driver mutations may be excluded from a TMB calculation. In certain examples. “tumor mutational burden” or “TMB score” refers to the number of somatic mutations in a tumor's genome and/or the number of somatic mutations per area of the tumor's genome. In some embodiments, TMB, as used herein, refers to the number of somatic mutations per megabase (Mb) of DNA sequenced. In some embodiments, germline (inherited) variants are excluded when determining TMB, given that the immune system has a higher likelihood of recognizing these as self. In addition, germline variants do not reflect the biology of somatic mutation for the purposes of TMB determinations. In various cases, driver mutations are excluded from a TMB calculation.

In some cases, the features 118 include the presence, amount, type, or any combination thereof, of one or more hotspot mutations. Hotspots, for instance, can refer to loci in the genome of the subject 102 and/or the lesion 104 that are prone to mutation. Examples of hotspots include CpG islands, microsatellites, centromeric DNA, telomers, subtelomeric regions, common fragile sites, palindromic AT-rich repeats (PATRRs), G-quadruplexes, R-loops, and the like.

Hotspot mutations give rise to oncological outcomes. PhyloP, SIFT, Grantham. COSMIC and PolyPhen-2 are in silico tools that can be used to assess pathogenicity of identified variants. Exemplary hotspot genes and mutations include EGFR exon 19 activating mutation, EGFR exon 19 deletion, EGFR exon 19 insertion, EGFR exon 19 sensitizing mutation, EGFR exon 20 activation mutation, EGFR exon 20 insertion, EGFR G719 mutation, EGFR L858R mutation, EGFR L861 mutation, EGFR S768 mutation, EGFR T790M mutation, C797 mutation, KIT activating mutation, KRAS activating mutation, MET activating mutation, NRAS activating mutation, PMS2 promoter mutations, among many others, Hotspot mutations also occur in the following genes: AKT2, BRCA1, BRCA2, ERC1, NSD1, POLH, PPMIG, PTEN, RAD18, RAD51, RAD51B, RB1, TERT, TP53, TP53Bp1, ALK, ARMTI, ATAD5, ATG7, ATIC, AXL, BIRC6, BRD3, BRD4, CAPRIN1, CCAR2, CCDC6, CDK5RAP2, CHD9, CIT, CTNNB1, CUL1, EBF1, EIF3E, HIP1, HMGA2, IRF2BP2, NOTCH1, NOTCH4, NPM1, OFD1, TACC1, TACC3, TERF2, TMEM106B, UBE2L3, USP10, WRDR48, YAP1, ZEB2, and ZMYND8.

The features 118, in particular examples, include the presence, amount, type, or any combination thereof, of one or more aneuploidy events. For instance, the features 118 may indicate whether the subject 102 and/or the lesion 104 includes one or more extra chromosomes (e.g., greater than a pair of 23 chromosomes) or one or more missing chromosomes (e.g., less than the pair of 23 chromosomes).

In some implementations, the features 118 include a tumor purity of the sample 106. In various implementations, the tumor purity represents an amount of the nucleic acid molecules 108 that originate from a tumor (e.g., the lesion 104) with respect to a total amount of the nucleic acid molecules 108 in the sample 106. Tumor purity can be estimated, for instance, based on a presence or amount of somatic copy-number alterations (SCNA), single-nucleotide variants (SNVs), minor allele frequency (MAF), or any combination thereof, observed with respect to the sequence read data 114.

In some cases, the features 118 include additional biomarker data. That is, the features 118 may include non-genomic features. For instance, features 118 may include data indicating at least one of a histological and/or immunohistological image of the sample 106 or another sample of the lesion 104, a genomic alteration, or a viral status of the subject 102 and/or lesion 104. The additional biomarker data may be generated based on the sample 106, medical images, or other samples obtained from the subject 102. In some cases, the additional biomarker data includes an image of a stained section of the lesion 104. For instance, the stained section is stained with hematoxylin and eosin (H&E) and/or at least one immunostain.

To categorize the cancer, a predictive model 120 is configured to generate a prognostic classification 122 based on the features 118. The predictive model 120, for example, may include one or more mathematical and/or computer-based models that are configured to predict the prognostic classification 122 based on the features 118. For instance, the predictive model 120 may include a regression model, threshold rule, confidence interval, or other type of statistical model capable of categorizing the cancer based on the features 118. In various cases, the predictive model 120 includes at least one classifier configured to generate the prognostic classification 122 based on the features 118.

In various implementations, the predictive model 120 includes at least one trained ML model configured to output the prognostic classification 122 in response to receiving the features 118 in input data. For example, parameters of the ML model(s) may have been previously optimized based on training data including features of individuals within a population omitting the subject 102. For instance, the ML model(s) was trained using an unsupervised or semi-supervised learning technique, wherein the parameters were optimized to categorize (e.g., cluster) the features of the population. In some cases, the ML model(s) was trained using a supervised learning technique, wherein the training data further included ground truth prognostic classifications of the individuals in the population, such that the parameters were optimized to minimize a loss between predicted prognostic classifications generated by the ML model(s) based on the features of the population and the ground truth prognostic classifications of the cancers experienced by the individuals in the population. To increase training robustness, the population represented by the training data may include individuals without cancer, as well as individuals with a variety of cancer types and metastasis states. Various types of ML models can be included in the predictive model 120, such as a neural network (e.g., a convolutional neural network (CNN)), a nearest-neighbor model, a regression analysis model, a clustering model, a principal component analysis model, a gradient boosting model, a random forest, or any combination thereof. In some cases, the predictive model 120 includes a hybrid model, that includes multiple types of ML models. For instance, the predictive model may include a CNN and a clustering model.

In particular examples, the predictive model 120 includes a clustering model 121. In various implementations, the clustering model 121 is pre-trained based on training data that includes population features. According to various implementations, the population features include genomic features and/or additional biomarker data of the population. In some cases, the population features further include one or more known prognostic features and/or prognostic classifications of the population. In various implementations, at least one computing device is configured to cluster the population features. The clustering model 121, for instance, stores, includes, or otherwise indicates the determined clusters.

In various examples, the population characteristics are defined in a multi-dimensional feature space. In various cases, the feature space has n dimensions, wherein n corresponds to the number of feature types included in the population feature. For example, one dimension may correspond to MMRD probability scores of the members of the population, another dimension may refer to the copy number states of the members of the population, and so on. In various cases, data objects representing the population features of the population are plotted or otherwise defined in the feature space. In some examples in which n is greater than two, the data objects are projected onto an m-dimensional feature space using multi-dimensional scaling, wherein m is between 1 and n−1 (inclusive). Multi-dimensional scaling can be achieved using various techniques. For instance, multi-dimensional scaling can be performed using at least one of a statistical method (e.g., t-distributed stochastic neighbor embedding (t-SNE), uniform manifold approximation and projection (UMAP), representation learning (e.g. principal component analysis (PCA), independent component analysis (ICA), etc.), ML-based latent space learning (e.g. autoencoders, transformers, generative adversarial networks, etc.). Accordingly, in some cases, the data objects can be visualized in a Cartesian coordinate system.

Within the feature space (whether it has two or more than two dimensions), the data objects are separated from each other by distances (also referred to as “distance metrics”). Various types of distances can be utilized in implementations of the present disclosure. For example, the distances may include Euclidian distances, Manhattan distances, Hamming distances, Minkowski distances, Chebyshev distances, or any combination thereof.

Various clustering techniques can be utilized to generate the clustering model 121. For instance, the clusters may be generated using k-means clustering, density-based clustering, centroid-based clustering, spectral clustering, distribution-based clustering, hierarchical clustering, or any combination thereof. In some implementations, the clustering model 121 is generated by performing hierarchal clustering on the data objects representing the population features. In various cases, the clusters include two or more data objects that are within proximity of each other (e.g., within a predetermined distance of one another) in the feature space. For instance, a cluster may include two or more data objects that are within a predetermined distance (e.g., Euclidian distance) of one another in the feature space. In some implementations, a data object is included in a cluster if the data object is within an appropriate distance of a linkage criterion representing one or more data objects that are already defined within the cluster. Various implementations of the present disclosure utilize one or more linkage criteria, such as a single-linkage criterion, a complete-linkage criterion, an average-linkage criterion (e.g., a weighted average criterion, an unweighted average criterion), a centroid-linkage criterion, a median linkage criterion, a Ward linkage criterion, a minimum error sum of squares criterion, a min-max criterion, a Hausdorff linkage criterion, a medoid linkage criterion, a minimum energy clustering criterion, or any combination thereof.

In some cases, agglomerative clustering is used to generate the clusters. For example, initially, each data object is defined within the feature space without clustering. Subsequently, pairs of adjacent data objects may be clustered together. In some examples, the process of generating a cluster based on independent data objects in a feature space, or of adding a data object to an existing cluster, may be referred to as “merging.”

In some examples, divisive clustering is used to generate the clusters. For example, the data objects may be defined into a single cluster in the feature space. Subsequently, the single cluster may be divided into multiple clusters. In some instances, the process of dividing a preliminary cluster into multiple subsequent clusters, or of removing a data object from a cluster, may be referred to as “splitting.”

In various cases, each cluster is defined according to a boundary (also referred to as a “border”). In some implementations, data objects outside of the boundary of a cluster are not part of the cluster. Data objects inside of the boundary of the cluster are part of the cluster. Depending on the data objects, the linkage criterion, the feature space, and other characteristics of the training data, the clusters may have irregular shapes within the feature space. In various cases, the clustering model 121 includes the boundaries of the clusters generated based on the data objects defined by the population features.

According to various cases, each cluster in the clustering model 121 is associated with one or more prognostic features. In some cases, at least one prognostic characteristic is defined in at least one dimension of the feature space, such that the clusters are defined according to the prognostic feature(s). In some examples, the population features used to define the clusters omit prognostic features. Once the clusters are generated based on non-prognostic features (e.g., genomic features and/or additional biomarker data), prognostic features associated with the clusters are subsequently determined. For example, an example cluster may be defined based on the data objects representing the non-prognostic population features of m members of the population, wherein m is an integer that is greater than one. In various cases, prognostic features of the m members of the population are determined. Common prognostic features of the population are determined. For example, if greater than a threshold number of the m members have a cancer that is resistant to a predetermined therapy, than resistance to the predetermined therapy may be associated with the example cluster. In various cases, each cluster may be labeled with, or otherwise associated with, one or more prognostic features. The one or more prognostic features associated with a given cluster form the prognostic classification associated with the cluster. In various cases, each cluster in the clustering model 121 is associated with a prognostic classification.

In some examples, defining prognostic classifications using unsupervised clustering has advantages over existing technologies. For example, clinical observations may be used to establish different prognostic classifications of a given disease. However, a single prognostic classification may be associated with different genomic features. By clustering the genomic features and associating the clusters with prognostic classifications, the single prognostic classification may be separated into multiple disease subtypes. With the benefit of identifying the multiple disease subtypes, which would not be otherwise apparent based on an analysis of prognostic features alone, care providers and researchers may potentially develop new interventions that target one of the disease subtypes, may perform additional analyses that update treatment and disease management for the distinct subtypes, or the like.

In various implementations, the cancer of the subject 102 is categorized by comparing the features 118 of the subject 102 to the clusters in the clustering model 121. The prognostic classification 122 is determined based on a comparison between the features 118 and the clusters in the clustering model 121. In various cases, a data object defined by the features 118 of the subject 102 is defined in the feature space of the clustering model 121. The clustering model 121, for instance, may determine that the data object is present within the boundary of a particular cluster that was previously defined based on the training data. In some cases, the clustering model 121 determines that the data object is associated with a particular cluster based on a distance between the data object and the particular cluster in the feature space. In some cases, the distance is at least one of a Euclidian distance, a Manhattan distance, a Hamming distance, a Minkowski distance, a Chebyshev distance, or any combination thereof. For instance, the clustering model 121 determines that the distance between the data object and the boundary and/or a centroid of the particular cluster is below a threshold distance. In some examples, the clustering model 121 classifies the cancer of the subject 102 into a classification associated with the particular cluster by determining that a distance between at least one data object corresponding to the population features in the cluster is below a threshold distance.

In various cases, the prognostic classification 122 of the sample 106 is generated. For example, the clustering model 121 may determine that the disease of the subject 102 is associated with one or more prognostic features associated with the cluster in which the features 118 belong. In various examples, the prognostic features may include predicted characteristics of the disease that is experienced by the subject 102, predicted symptoms (e.g., predicted chronic symptoms, such as heart disease, diabetes, high blood pressure, etc.; or predicted medical events, such as heart attack, stroke, pre-eclampsia, etc.) of the subject 102, predicted causes of the disease, or the like. For instance, the prognostic classification 122 includes one or more of a predicted disease of the subject 102; a predicted disease subtype of the subject 102; a predicted survivability of the subject 102; one or more predicted symptoms of the subject 102; a predicted effective therapy to treat the predicted disease of the subject 102; a predicted stage of the predicted disease of the subject 102; a predicted grade of the predicted disease of the subject 102; a predicted activity level of the subject 102 (e.g., a predicted Eastern Cooperative Oncology Group (ECOG) performance status of the subject 102); a predicted diabetes status of the subject 102; a predicted body mass index (BMI) of the subject 102; a predicted smoking history of the subject 102; a predicted breast density of the subject 102; a clinical trial that the subject 102 is predicted to qualify for; or a characteristic of the predicted disease of the subject. Accordingly, the prognostic features, and prognostic classification, of the cancer of the subject 102 can be determined based on the features 118.

In some implementations, the predictive model 120 is unable to conclusively categorize the disease of the subject 102. For example, the predictive model 120 may determine that the features 118 of the subject 102 do not fit within any of the previously defined clusters in the clustering model 121. In various cases, the predictive model 120 may output an indication that that the categorization of the disease is inconclusive.

A report generator 124 is configured to generate a report 126 based, at least in part, on the prognostic classification 122. The report 126, for example, includes consumable data that can inform the care provider 105 about the prognostic classification of the subject 102. In various implementations, the report 126 may indicate the results of additional analyses, such as the results of a histological study, whole transcriptome sequencing, cfRNA sequencing, whole exome sequencing, whole genome sequencing, a cancer (e.g., DNA) hotspot panel test, a DNA methylation test, a tumor mutational burden (TMB) test, a DNA fragmentation test, an RNA fragmentation test, a microsatellite instability (MSI) test, a tumor mutational burden (TMB) test, or a viral status test. The performance of such tests is within the ordinary skill of the art, with additional detail provided elsewhere herein. The report 126, for example, may include a genomic profile of the subject 102 based on various combinations of the above analyses and tests.

In some implementations, the report 126 indicates that a follow-up test of the subject 102 is indicated. For instance, in response to determining that the categorization of the disease is inconclusive, the report generator 124 may generate the report 126 to indicate that one or more additional tests (e.g., a histological study, genome sequencing, exome sequencing, additional DNA sequencing, RNA sequencing, transcriptome sequencing, etc.) should be performed in order to identify the cancer of the subject 102.

In various cases, the report 126 is output to a clinical device 128. For example, the report generator 124 transmits the report 126 to the clinical device 128. In various implementations, the clinical device 128 is a computing device that is operated by, owned by, or otherwise associated with the care provider 105. For instance, the clinical device 128 may be a desktop computer, a laptop computer, a smart phone, or some other computing device associated with the care provider 105. The clinical device 128, in various cases, outputs the report 126 to the care provider 105. In some cases, the clinical device 128 includes a display (e.g., a screen) that visually presents the report 126. In various cases, the clinical device 128 includes a speaker that outputs a sound indicative of the report 126. The clinical device 128, in various cases, may output the information in the report 126 using one or more output mechanisms or devices.

The care provider 105 may review the report 126 by interacting with the clinical device 128. The report 126, in various cases, may enhance the clinical decision-making of the care provider 105. For instance, the care provider 105 may prepare and/or administer a therapy to the subject 102 based on the report 126. According to various implementations, the care provider 105 may initiate the therapy and/or refer the subject 102 to another care provider to receive the therapy. In various cases, if the disease of the subject 102 is cancer, the care provider 105 may prescribe, recommend, or administer an anticancer agent for the subject 102.

In various implementations, the care provider 105 may develop a diagnosis and/or prognosis of the subject 102 based on the report 126. In various implementations, the care provider 105 may communicate information in the report 126 to the subject 102.

Although FIG. 1 is generally described with reference to categorizing cancer of the subject 102, implementations are not so limited. For example, similar techniques can be used to determine prognostic classifications of other types of diseases, such as diabetes, heart disease, cystic fibrosis, arthritis, muscular dystrophy, and the like.

FIG. 1 illustrates various elements that can be embodied in one or more computing devices. For example, at least a portion of the functions of the sequencer 112, the feature selector 116, the predictive model 120, the report generator 124, the clinical device 128, or any combination thereof, is performed by one or more processors in at least one computing device. Examples of computing devices include server computers, desktop computers, laptop computers, tablet computers, mobile phones, wearable devices, Internet of Things (IoT) devices, and the like. In various cases, instructions for performing at least a portion of the functions of these elements are stored in memory and/or in a non-transitory computer readable medium. The instructions, for instance, are executed by the processor(s).

FIG. 1 also illustrates various types of data. For example, the sequence read data 114, the features 118, the prognostic classification 122, the report 126, or any combination thereof, includes data. The various types of data illustrated in FIG. 1 may be stored, such as in memory or in non-transitory computer readable media. In various implementations, at least a portion of the data is transmitted or otherwise output by one or more computing devices. For example, a computing device may transmit one or more communication signals to another computing device, wherein the communication signal(s) encode at least a portion of the data. Examples of communication signals include electromagnetic signals, optical signals, ultrasonic signals, optical signals, and electrical signals. For example, communication signals can be transmitted wirelessly and/or in a wired fashion. The communication signals, for instance, are transmitted over one or more wireless channels and/or one or more wired channels (e.g., optical cabling, electrical cabling, etc.). In various cases, the communication signal(s) are transmitted over one or more communication networks. A communication network, for instance, may be defined according to one or more physical channels, such as one or more frequency spectra. In some cases, a communication network is defined according to one or more communication protocols and/or standards. Examples of communication networks include fiber optic networks, Institute of Electrical and Electronics Engineers (IEEE) networks (e.g., WI-FI™ networks, WiMAX networks, BLUETOOTH™ networks, etc.), cellular networks (e.g., a 3rd Generation Partnership Project (3GPP) radio network, such as a Long Term Evolution (LTE) network, a New Radio (NR) network; or a cellular core network such as a 3rd Generation (3G) core, a 4th Generation (4G) core, a 5th Generation (5G) core, etc.), ultrasonic networks, and the like. In some cases, the data is broadcasted from one device to multiple other devices. In some cases, the data is unicasted from one device to another device. For instance, various forms of data described herein may be transmitted via a peer-to-peer (P2P) connection.

A particular example will now be described with reference to FIG. 1. The subject 102 may present to a clinical environment with symptoms consistent with endometrial cancer. For example, the subject 102 may report experiencing gynecological bleeding to the care provider 105. In various cases, the sample 106 is a blood sample obtained from the subject 102. The nucleic acid molecules 108, for instance, include cfDNA such as ctDNA. In various examples, the nucleic acid molecules 108 include fragments. In some examples, the nucleic acid molecules 108 include cellular DNA. The sequence read data 114 generated by the sequencer 112 is indicative of the identity, order, and position of the DNA in the sample 106.

In various implementations, the feature selector 116 generates the features 118 based on the sequence read data 114. For example, the feature selector 116 may determine whether the sequence read data 114 is indicative of one or more pathogenic variants in at least one of POLE, TP53, CTNNB1, LICAM, PTEN, ERBB2, PMS2, MSH2, MSH6, MLH1, an ER gene, or a PR gene. In various cases, the feature selector 116 may determine an MMRD probability score of the sample 106 by analyzing the sequence read data 114. In various cases, the feature selector 116 determines a copy number state of at least one genetic loci by analyzing the sequence read data 114.

In various cases, a data object representing the features 118 is identified in a multi-dimensional feature space of the clustering model 121. In various cases, the dimensions of the feature space respectively represent the type of features included in the features 118. For instance, the feature space may include at least one dimension representative of amount and/or types of pathogenic variants in at least one of POLE, TP53, CTNNB1, LICAM, PTEN, ERBB2, PMS2, MSH2, MSH6, MLH1, the ER gene, or the PR gene. In some examples, the feature space includes a dimension representative of MMRD probability score. In various cases, the feature space includes a dimension representative of copy number state.

Multiple clusters are further defined within the feature space of the clustering model 121. Each cluster may be assigned, or otherwise associated with, a prognostic classification. In various cases, the clusters of the clustering model 121 are previously defined based on features (e.g., genomic and/or biomarker features) of a population of individuals omitting the subject 102. The population may include different people with different types or subtypes of endometrial cancer.

The Cancer Genome Atlas (TCGA) Consortium (see, e.g., Levine et al., Nature 497, 67-73 (2012)) defines four potential prognostic classifications of endometrial cancer. Each of the four prognostic classifications may be associated with associated effective therapies, likelihood of recurrence, survivability, and other prognostic characteristics. From best to worst prognosis, the prognostic classifications include (1) a first group defined by POLE exonuclease deficiency, resulting in ultramutation-level TMB (also referred to as “POLE ultra-mutated”); (2) a second group defined by MMR deficiency, with high MSI fraction (e.g., an MSI that is above a threshold fraction), resulting in numerous characteristic mutations (also referred to as “microsatellite instability hypermutated”; (3) a third group defined by a lack of extensive copy number alterations (also referred to as “copy number low”); and (4) a fourth group defined by a substantial number of copy number alterations, associated TP53 mutations, and, frequently, serous-like histological features. In general, cancers associated with the first and second groups are responsive to known treatments, such as chemotherapies. Cancers associated with the fourth group are often recurrent, resistant to known treatments, and have a high likelihood of metastasis. The third group, however, is a “catch-all” group that includes cancers that are not clearly delineated into the first group, the second group, and the fourth group. Due to the disparate risks for morbidity and mortality associated with different types of endometrial cancer, it is highly clinically relevant to determine a patient's prognostic classification quickly after an initial diagnosis of endometrial cancer.

In some examples, at least four clusters are included in the clustering model 121, which respectively correspond to the four prognostic classifications defined by the TCGA. However, in some cases, the clustering model 121 includes additional clusters. For instance, by clustering the features of the population, the clustering model 121 may identify multiple distinct clusters in the feature space that correspond to the third, “catch-all” group. For instance, the third group may be divided into a first cluster corresponding to what is defined as Type 3A endometrial cancer and a second cluster corresponding to what is defined as Type 3B endometrial cancer. Further, once the clusters are separated, it may be determined that Type 3A endometrial cancers tend to be resistant to a particular immunotherapy, whereas Type 3B endometrial cancers tend to be responsive to the particular immunotherapy.

In this example, the data object representing the features 118 of the subject 102 is located within the boundary of the Type 3B endometrial cancer cluster within the feature space. The prognostic classification 122, for instance, reflects that the subject 102 is predicted to have Type 3B endometrial cancer. The report generator 124, in various cases, generates the report 126 to reflect that the subject 102 has Type 3B endometrial cancer. In addition, the report 126 may indicate that the particular immunotherapy is predicted to be effective at treating the endometrial cancer of the subject 102. Accordingly, the report 126 may assist the care provider 105 with diagnosing, treating, and managing the endometrial cancer of the subject 102.

FIG. 2 illustrates an example of hierarchical clustering of data objects representing population features. In particular, FIG. 2 illustrates a two-dimensional feature space 200, in which a first, horizontal dimension represents MMRD probability score and a second, vertical dimension represents copy number state. Although only two dimensions are illustrated in FIG. 2, implementations are not so limited. In various implementations, three-or-more dimensional feature spaces can be utilized.

Various population data objects 202 are presented within the feature space. The population data objects 202 represent observed features of individuals in a population. In various cases, an example population data object among the population data objects 202 represents an observed MMRD probability score and copy number state of a single individual within the population. The population, in various cases, includes people with a disease and without a disease. In some cases, the population includes individuals with different types and/or subtypes of diseases. For instance, the population may include people without cancer, with different classifications of endometrial cancer, with bladder cancer, with breast cancer, and the like. In some cases, the population includes people with cancers at different stages of progression. In various implementations, the features of the individuals in the population are determined by obtaining samples from the individuals.

In various implementations, the population data objects 202 are clustered using agglomerative hierarchical clustering. In the example of FIG. 2, the geometric distance in the feature space between population data objects is utilized as the distance for clustering. For example, the closest pairs of the population data objects 202 are initially grouped in preliminary clusters, the closest preliminary clusters are combined, and the process is repeated until small clusters 204 of the population data objects 202 are generated. In various cases, the small clusters 204 are grouped into medium clusters 206. The medium clusters 206 may be grouped into a large cluster 208 that encircles all of the population data objects 202.

In some cases, the population data objects 202 are clustered using divisive hierarchical clustering. For example, the large cluster 208 encircling all of the population data objects 202 is initially defined. Then, the population data objects 202 are divided into medium clusters 206 in view of the geometric distance between the population data objects 202. Further, the medium clusters 206 may be divided into the small clusters 204.

In various implementations, the small clusters 204 or the medium clusters 206 are defined as the clusters for the purposes of future prognostic classifications of subjects. In some implementations, the small clusters 204 are selected as the appropriate clusters due to the relative proximity of the population data objects 202 within the feature space. In some cases, the medium clusters 206 are selected due to an analysis of the prognostic features of the individuals whose population data objects 202 are grouped within the medium clusters 206. For instance, it may be observed that all individuals whose population data objects 202 are grouped within one of the medium clusters 206 have a first cancer subtype, and it may be observed that all individuals whose population data objects 202 are grouped within the other of the medium clusters 206 have a second cancer subtype. In various cases, the clusters utilized for classification are labeled according to prognostic features of the individuals whose population data objects 202 are grouped in the clusters.

According to some examples, a sample is obtained from a subject and analyzed. For example, the MMRD probability score and copy number state of the sample is determined. A data object representing the MMRD probability score and copy number state of the sample is represented within the feature space. Then, data object is compared to the clusters utilized for classification. For example, the data object may be determined to be within the boundary of one of the clusters. Based on the position of the data object within the feature space in comparison to the clusters, the subject and/or sample is classified. For example, the subject is assigned a prognostic classification based on the prognostic features associated with the cluster in which the data object is located. In a particular instance, the cluster is associated with a particular cancer subtype that is resistant to a first therapy, but can be effectively treated using a second therapy. The prognostic classification of the subject indicates that the second therapy is predicted to be therapeutically effective. Accordingly, a care provider may enable treatment of the subject using the second therapy.

FIG. 3 illustrates an example of comparing subject data objects to a previously defined cluster for the purpose of prognostic classification. Specifically, the cluster is defined based on various population data objects 302 that are defined within a feature space. A boundary 304 defines the edge of the cluster.

A first subject data object 306 is further defined within the feature space. As noted in FIG. 3, the first subject data object 306 is located within the boundary 304 of the cluster. Accordingly, the first subject data object 306 may be classified with a prognostic classification associated with the cluster.

Further, a second subject data object 308 is defined within the feature space. Unlike the first subject data object 306, the second subject data object 308 is defined outside of the boundary 304 of the cluster. Nevertheless, in some examples, the second subject data object 308 may be defined as being associated with the cluster based on one or more distances between the cluster and the second subject data object 308.

For instance, the second subject data object 308 may be classified as being within the cluster based on a centroid distance 310 extending between a centroid 312 of the cluster and the second subject data object 308. The centroid 312 may be the geometric center of the cluster. In some cases, the centroid 312 represents the arithmetic mean position of the data objects 302 defined within the cluster. If the centroid distance 310 is less than a threshold distance, for instance, then the second subject data object 308 may be determined to be part of the cluster.

In some cases, the second subject data object 308 may be classified as being within the cluster based on an object distance 314 extending between one of the population data objects 302 and the second subject data object 308. For instance, if the object distance 314 is less than the threshold distance, then the second subject data object 308 may be determined to be part of the cluster. Although a single object distance 314 is illustrated in FIG. 3, implementations are not so limited. For example, the second subject data object 308 may be defined as within the cluster based on multiple object distances 314 corresponding to multiple population data objects 302 in the cluster. In some cases, the second subject data object 308 is defined as within the cluster based on a comparison between the threshold distance and an arithmetic mean of distances between the second subject data object 308 and two, three, four, or more population data objects 302 within the cluster.

FIG. 4 illustrates an example environment 400 for training and utilizing a predictive model 402 to determine prognostic classifications of one or more diseases. The predictive model 402, for instance, is the predictive model 120 described above with reference to FIG. 1. In various implementations, the predictive model 402 includes a clustering model 404, which may include one or more ML models. A trainer 406, for instance, is configured to optimize various parameters 408 of the clustering model 404 based on training data 410.

The training data 410 includes example features 412. The example features 412, in various cases, include genomic features that are obtained based on sequencing of DNA and/or RNA of individuals within a population 416. According to some examples, the example features 412 include additional biomarker data of the population 416. In some implementations, the example features 412 include prognostic features, which indicate prognoses of the population 416. According to various examples, the population 416 includes a variety of different types of individuals. The population 416 may include people with one or more diseases and without the disease(s). In some cases, the population 416 includes people with different demographics (e.g., age, sex, etc.). In some cases, the population 416 includes people with different diseases and/or disease subtypes.

The clustering model 404 includes one or more ML models. In various cases, the clustering model 404 indicates one or more clusters of the example features 412 indicated in the training data 410. In various implementations, the clusters are defined based on data objects representing the example features 412 of the training data 410. The clusters may be generated using an unsupervised learning technique. In various cases, the shape, orientation, position, or any combination thereof, of the clusters are defined according to the parameters 408.

In some cases, the clustering model 404 maps various data points (e.g., training data) to a feature space. Based on the proximity of groups of those data points in the features pace, one or more “clusters” are defined. An additional data point may be classified according to one or more of the clusters based on its proximity to the clusters (e.g., a center of the clusters, a boundary of the cluster, etc.). Examples of clustering models include k-means clustering, mean-shift clustering, expectation-maximization (EM) clustering, and agglomerative hierarchical clustering. The parameter(s) 408, for example, include a threshold proximity within which a new data point is classified within a cluster, a density of points used to define a cluster, and the like.

The clustering model 404, for example, includes a random forest. The random forest, for instance, includes multiple decision trees that classify data in an ensemble fashion. In various implementations, the decision trees are defined by the parameter(s) 408.

In some implementations, the clustering model 404 include a nearest-neighbor model. One example of a nearest-neighbor model includes a k-nearest neighbor model. For example, a nearest-neighbor model defines various “neighbors,” which are points within a feature space, with associated class labels. When a new data point is mapped to the feature space, the new data point is classified based on the proximity (e.g., Euclidian distance, Manhattan distance, Minkowski distance, etc.) of its “neighbors” to the new data point as well as their associated classes. In some cases, the new data point is classified as belonging to a particular class if greater than a threshold number of neighbors within a threshold distance of the new data point are members of the class. For instance, the parameters 408 may include k (e.g., the number of neighbors compared to the new data point), the threshold distance, and so on.

In various examples, the clustering model 404 include a principal component analysis model. In various implementations, a principal component analysis defines a collection principal components of unit vectors within a coordinate space based on a data set (e.g., training data 410). The model, for example, is an orthogonal linear transformation of the data set. Various weights of the model, for example, are included in the parameter(s) 408.

In some cases, the predictive model 402 includes one or more additional model types. For instance, the predictive model 402 includes an artificial neural network. An artificial neural network includes various layers that respectively process input data. For example, an artificial neural network includes an input layer, one or more hidden layers, and an output layer. The input layer performs a pre-processing operation on the input data. The hidden layer(s) may perform various processing operations on the output from the input layer. The output layer, in various cases, processes the output from the hidden layer(s). Each layer, in some cases, includes one or more nodes, which are defined by individual operations. In various cases, the hidden layer(s) include nodes that are connected to each other in parallel and/or series. Examples of artificial neural networks include feedforward neural networks, multi-layer perceptrons (MLPs), convolutional neural networks (CNNs), and backpropagation models. In various implementations, the operations performed by the layers and/or nodes within an artificial neural network included in the predictive model 402 is defined according to the parameters 408. For example, the predictive model 402 includes data representing weights, thresholds, filters, kernels, or other data objects that are utilized to perform operations of the predictive model 402.

In various cases, the predictive model 402 include a regression analysis model. The regression analysis model, for example, is defined by a regression function that defines relationships between one or more independent variables and one or more dependent variables. The regression function may further define one or more unknown parameters that define a relationship between the independent and dependent variables. In various implementations, the unknown parameters and/or the type of regression function (e.g., linear, quadratic, etc.), is defined according to data stored in the predictive model 402.

The predictive model 402, in some implementations, includes a gradient boosting model. For example, the gradient boosting model is defined as a collection of prediction models (e.g., decision trees) that iteratively classify observed data. In various cases, the type of prediction model, weights in the prediction models, and the like, are defined by the predictive model 402.

In some examples, the outputs from the additional models in the predictive model 402 are input into the clustering model 404. For example, the predictive model 402 may include a CNN that receives immunohistological images of tumors as inputs and produces metrics indicative serous feature severity as outputs. The metrics produced by the CNN, for instance, may be used as features plotted within the multi-dimensional feature space of the clustering model 404.

In various implementations of the present disclosure, the trainer 406 is configured to generate and/or optimize the parameters 408 based on the training data 410. In various implementations, the trainer 406 is configured to identify patterns in the training data 410 using an unsupervised learning technique. For example, the trainer 406 plots data objects representing the example features 412 into a multi-dimensional feature space. In some cases, the trainer 406 defines clusters of the data objects using any clustering technique described herein. The parameters 408, for instance, define the clusters of the data objects. In various cases, the trainer 406 optimizes the parameters 408 iteratively based on the entire set of the training data 410.

In some examples, the trainer 406 is further configured to correlate the clusters with prognostic classifications. For example, in some cases, the example features 412 include prognostic features. Each cluster may be therefore associated with one or more types of prognostic features based on their location within the feature space. In some examples, the trainer 406 receives the prognostic features of the population 216 after the clusters in the clustering model 404 have been defined. The trainer 406 may determine prognostic features that are correlated with the defined clusters. In some cases, the trainer 406 determines the prognostic features based on prognostic outcomes in datasets reflecting individuals that would (or that do) fit within the defined clusters. For instance, the trainer 406 may rely on datasets from the CGDB, clinical trial data, or research datasets for outcomes (e.g., survivability, chance of remission, etc.) as well as other derived metrics associated with prognosis (e.g., POLE signatures, etc.). In various cases, the trainer 406 labels the clusters based on the associated prognostic features. The collection of prognostic features associated with a given cluster may be representative of a prognostic classification of that cluster.

In various implementations, the optimization of the parameters 408 enables the predictive model 402 to identify predictive attributes of the example features 412 that are indicative of their corresponding clusters. For instance, the predictive model 402 may determine that a particular combination of variants represented in the example features 412 and associated with a cluster in the clustering model 404 is highly correlated with a type of breast cancer having low survivability. The predictive model 402 may therefore classify diseases (e.g., cancers) based on characteristics and/or subjects outside of the example features 412 by recognizing or otherwise identifying the predictive attributes.

Once the parameters 408 are optimized, the predictive model 402 may be ready to classify a new set of data. For example, the predictive model 402 may receive input data including features 418 of a subject. The features 418, for instance, may include one or more of the predictive attributes. The predictive model 402 may perform various operations on the input data based on the clustering model 404 and the optimized parameters 408. In various cases, the predictive model 402 outputs output data including one or more prognostic classifications 420 based on the features 418. The prognostic classification(s) 420, for instance, include one or more predicted categories of a cancer experienced by the subject.

FIG. 5 illustrates an example of training data 500 utilized to train one or more ML models. For example, the training data 500 may be the training data 410 described above with reference to FIG. 4.

The training data 500, in various cases, may represent m samples, wherein m is a positive integer. In some cases, the m samples are respectively obtained from m individuals within a population, although implementations are not so limited. For example, in some cases, multiple samples may be obtained from the same individual at different times.

The training data 500 includes first to mth example features 502-1 to 502-m. For example, the first to mth example features 502-1 to 502-m include features derived from DNA and/or RNA (e.g., cfDNA, ctDNA, mRNA, or the like) in the respective m samples. According to some cases, the firs to mth example features 502-1 to 502-m include additional biomarker data associated with the m individuals in the population. In some cases, the first to mth example features 502-1 to 502-m include prognostic features of individuals from which the m samples are obtained.

In various cases, the first to mth example features 502-1 to 502-m can be converted into data objects for the purposes of clustering. For example, the first to mth example features 502-1 to 502-m can be respectively converted to first to mth data objects that can be distributed in a feature space. In various cases, clusters representing the first to mth example features 502-1 to 502-m can be determined and used to generate or identify prognostic classifications of a subject.

FIG. 6 illustrates an example report 600 summarizing predicted categories of a cancer of a subject. In various cases, the report 600 is the report 126 described above with reference to FIG. 1. The report 600, for instance, may be displayed to a patient and/or care provider. In some cases, the report 600 is generated based on features of a sample (e.g., a liquid biopsy sample) obtained from the subject.

The report 600 includes a tissue origin 602 of the cancer. The tissue origin 602, for instance, indicates a histological tissue type 604, a primary site 606, cell subtype 607, or any combination, of the cancer.

In various cases, the report 600 includes one or more therapy indicators 608. For instance, the therapy indicator(s) 608 convey whether the cancer is predicted to be resistant to one or more predetermined therapies and/or whether the cancer is predicted to be responsive to one or more predetermined therapies.

In some examples, the report 600 includes one or more prognostic indicators 610. The prognostic indicator(s) 610, for instance, indicate a prognosis of the subject in 6iew of the categorized cancer. For example, the prognostic indicator(s) 610 may indicate a survivability, a recoverability, a quality-of-life indicator, or other information indicative of the prognosis of the subject.

The report 600 may include a trial qualification 612 of the subject. The trial qualification 612, for instance, indicates whether the subject is predicted to qualify for a predetermined clinical trial.

The report 600, in various implementations, includes a metastasis profile 614 of the subject. The metastasis profile 614, for instance, indicates a likelihood that the cancer will metastasize (e.g., at a particular point in time), one or more tissues in which the cancer is predicted to metastasize, or the like.

In various cases, the report 600 includes recommended follow-up tests 616. For example, the report 600 may include a recommendation to perform whole genome sequencing on the subject, particularly in cases if the cancer cannot be categorized above a threshold certainty.

The report 600 may include a genomic profile 618 of the subject. In various cases, the genomic profile 618 includes or is generated based on the results of non-fragmentomic analyses of the subject.

FIG. 7 illustrates an example process 700 for clustering population features for the purposes of prognostic classification. The process 700 may be performed by an entity that includes at least one of the sequencer 112, feature selector 116, predictive model 120, clustering model 121, report generator 124, clinical device 128, predictive model 402, clustering model 404, trainer 406, or any combination thereof.

At 702, the entity identifies population features of individuals in a population. In various cases, the population features include genomic features of the individuals. According to some examples, the entity identifies data that includes sequence read data associated with the nucleic acid molecules. The nucleic acid molecules, for instance, may include RNA and/or DNA in samples obtained from the individuals. In some cases, at least a portion of the individuals have at least one disease (e.g., endometrial cancer). In various cases, the samples are obtained from lesions. In some cases, the samples include liquid biopsy samples and/or a tissue biopsy samples. According to some examples, the entity identifies the data by sequencing the nucleic acid molecules. In some cases, the entity receives the data from a sequencer that sequences the nucleic acid molecules. In some cases, the data is indicative of full genomes of the samples, RNA transcriptomes of the samples, whole exomes of the samples, or predetermined panel of genes of the samples.

In various examples, the features include MMRD probability scores of the samples. The MMRD probability scores may indicate a likelihood that one or more MMR genes in the genomes of the samples are nonfunctional or otherwise deficient. For instance, the MMRD probability score is indicative of at least one pathogenic variant in at least one MMR gene, a methylation status of the at least one MMR gene, or a methylation status of at least one promoter associated with the at least one MMR gene. In various cases, the MMRD probability score is indicative of a functional deficiency in at least one MMR gene, which may be related based on the presence of one or more variants in the MMR gene(s). According to some examples, the MMRD probability score is generated using a predictive model (e.g., at least one ML model). For instance, the entity may determine characteristics of the sequence read data that are associated with MMRD (which may include, for instance, the presence of one or more variants and/or a methylation status of an MMR gene, a promoter associated with an MMR gene, or an enhancer associated with an MMR gene), input the characteristics into the predictive model, and receive the MMRD probability score as an output of the predictive model.

In some cases, the features include copy number states of the samples. For example, the copy number states are of at least one genetic locus of the samples. In some cases, a copy number refers to a number of copies of a sequence present at a given genetic locus of the sample. The copy number states, in various cases, indicate an amount and/or type of one or more copy number respectively associated with one or more genetic loci of the samples.

In particular examples, the copy number state is generated by determining a minor allele coverage ratio and a major allele coverage ratio for multiple genetic loci indicated in the data. In various cases, the data is representative of a genome of the sample. In some cases, the genome indicated by the data is divided into genomic segments, such as based on the minor allele coverage ratio, the major allele coverage ratio, or a total coverage ratio. Input data for at least one model (e.g., at least one copy number grid model) can be generated based on the minor allele coverage ratio and the major allele coverage ratio. For instance, the input data includes, for each of a plurality of genetic loci. (i) a difference between the major allele coverage ratio and the minor allele coverage ratio, (ii) a sum of the major allele coverage ratio and the minor allele coverage ratio, or both. In various cases, each model is associated with a different copy number, or range of copy numbers. According to some cases, each model is initiated based on tumor purity estimates and/or tumor ploidy estimates of the sample, which may impact copies of various sequences indicated by the sequence read data. In various implementations, outputs of the models are compared, and a single model is selected based on the outputs. For example, the model associated with the predicted copy number for each segment of each genetic loci is determined. Accordingly, the copy number state can be determined for the sample.

In some examples, the features include the presence of pathogenic variants in one or more genes. For instance, the genes may include at least one of POLE, TP53, CTNNB1, LICAM, PTEN, ERBB2, PMS2, MSH2, MSH6, MLH1, at least one ER gene, or at least one PR gene. The features, in some examples, include fraction unstable scores.

According to various instances, the features include a mutation signature. According to some cases, the features include a TMB score. In some examples, the features indicate a presence and/or amount of hotspot mutations in the samples. In some cases, the features indicate a presence and/or amount of aneuploidy events in the samples.

In some implementations, the features include non-genetic biomarker data. For example, the features may include images of the samples, such as histological images of the samples. In some cases, the image represents photographs of stained portions of the samples, which may be stained hematoxylin and eosin (H&E) and/or at least one immunostain.

At 704, the entity maps the population features onto a multi-dimensional feature space. In various examples, the dimensions of the feature space correspond to the types of population features identified at 702. For example, one dimension of the feature space can correspond to MMRD probability score, one dimension can correspond to copy number state, and so on. In various cases, the entity maps data objects corresponding to the population features onto the feature space. For example, a single data object may correspond to the features of a single individual within the population.

At 706, the entity determines clusters of the population features. In various cases, the entity performs k-means clustering, density-based clustering, centroid-based clustering, spectral clustering, or distribution-based clustering on the data objects representing the population features. In some cases, the entity performs agglomerative or divisive hierarchical clustering on the population features mapped within the feature space. According to some cases, the entity further determines prognostic classifications associated with the clusters of the population features. For example, the entity may correlate the clusters with associated disease subtypes of the individuals within the population. In some cases, a cluster may be associated with a prognostic feature if greater than a threshold (e.g., 90%, 95%, 99%, etc.) of the individuals whose data objects are included in the cluster have a common prognostic feature. One or more prognostic features associated with a cluster may form a prognostic classification associated with the cluster.

FIG. 8 illustrates an example process 800 for performing prognostic classifications using clustering. The process 800 may be performed by an entity that includes at least one of the sequencer 112, feature selector 116, predictive model 120, clustering model 121, report generator 124, clinical device 128, predictive model 402, clustering model 404, trainer 406, or any combination thereof.

At 802, the entity identifies data indicative of sequences of nucleic acid molecules derived from a subject. According to some examples, the data includes sequence read data associated with the nucleic acid molecules. The nucleic acid molecules, for instance, may include RNA and/or DNA in a sample obtained from the subject. According to some examples, the subject has a lesion. In some cases, the subject has cancer, such as endometrial cancer. For instance, the lesion is an endometrial tumor. In various cases, the sample is obtained from the lesion. In some cases, the sample includes a liquid biopsy sample and/or a tissue biopsy sample. According to some examples, the entity identifies the data by sequencing the nucleic acid molecules. In some cases, the entity receives the data from a sequencer that sequences the nucleic acid molecules. In some cases, the data is indicative of a full genome of the sample, an RNA transcriptome of the sample, a whole exome of the sample, or a predetermined panel of genes of the sample.

At 804, the entity identifies features based on the data. In various examples, the features include an MMRD probability score of the sample. The MMRD probability score may indicate a likelihood that one or more MMR genes in a genome of the sample are nonfunctional or otherwise deficient. For instance, the MMRD probability score is indicative of at least one pathogenic variant in at least one MMR gene, a methylation status of the at least one MMR gene, or a methylation status of at least one promoter associated with the at least one MMR gene. In various cases, the MMRD probability score is indicative of a functional deficiency in at least one MMR gene, which may be related based on the presence of one or more variants in the MMR gene(s). According to some examples, the MMRD probability score is generated using a predictive model (e.g., at least one ML model). For instance, the entity may determine characteristics of the sequence read data that are associated with MMRD (which may include, for instance, the presence of one or more variants and/or a methylation status of an MMR gene, a promoter associated with an MMR gene, or an enhancer associated with an MMR gene), input the characteristics into the predictive model, and receive the MMRD probability score as an output of the predictive model.

In some cases, the features include a copy number state of the sample. For example, the copy number state is of at least one genetic locus of the sample. In some cases, a copy number refers to a number of copies of a sequence present at a given genetic locus of the sample. The copy number state, in various cases, indicates an amount and/or type of one or more copy number respectively associated with one or more genetic loci of the sample.

In particular examples, the copy number state is generated by determining a minor allele coverage ratio and a major allele coverage ratio for multiple genetic loci indicated in the data. In various cases, the data is representative of a genome of the sample. In some cases, the genome indicated by the data is divided into genomic segments, such as based on the minor allele coverage ratio, the major allele coverage ratio, or a total coverage ratio. Input data for at least one model (e.g., at least one copy number grid model) can be generated based on the minor allele coverage ratio and the major allele coverage ratio. For instance, the input data includes, for each of a plurality of genetic loci, (i) a difference between the major allele coverage ratio and the minor allele coverage ratio, (ii) a sum of the major allele coverage ratio and the minor allele coverage ratio, or both. In various cases, each model is associated with a different copy number, or range of copy numbers. According to some cases, each model is initiated based on tumor purity estimates and/or tumor ploidy estimates of the sample, which may impact copies of various sequences indicated by the sequence read data. In various implementations, outputs of the models are compared, and a single model is selected based on the outputs. For example, the model associated with the predicted copy number for each segment of each genetic loci is determined. Accordingly, the copy number state can be determined for the sample.

In some examples, the features include the presence of a pathogenic variant in one or more genes. For instance, the genes may include at least one of POLE, TP53, CTNNB1, LICAM, PTEN, ERBB2, PMS2, MSH2, MSH6, MLH1, at least one ER gene, or at least one PR gene. The features, in some examples, include a fraction unstable score.

According to various instances, the features include a mutation signature. According to some cases, the features include a TMB score. In some examples, the features indicate a presence and/or amount of hotspot mutations. In some cases, the features indicate a presence and/or amount of ancuploid events.

In some implementations, the features include non-genetic biomarker data. For example, the features may include an image of the sample, such as a histological image of the sample. In some cases, the image represents a photograph of a stained portion of the sample, which may be stained hematoxylin and cosin (H&E) and/or at least one immunostain.

At 806, the entity determines, using a clustering model, a prognostic classification of the subject based on the features. In various examples, the entity generates a data object based on the features of the subject. The data object may be mapped to a multi-dimensional feature space in which various clusters are defined. Based on the proximity of the data object to the clusters in the multi-dimensional feature space, the entity may determine a prognostic classification of the subject.

In particular cases, the entity determines a distance between a cluster and the data object. For example, the entity determines at least one of a Euclidian distance, a Manhattan distance, a Hamming distance, a Minkowski distance, or a Chebyshev distance between the data object and the cluster. In various cases, the distance is between the data object at least one additional data object representing one or more population features defined in the cluster, a centroid of the cluster, or a border of the cluster. In some implementations, the entity may determine that the data object is within the border of a cluster. If the distance between the data object and the cluster is below a threshold distance, or the data object is within the border of the cluster, the entity may determine that the data object is part of the cluster. In various cases, the entity determines that the subject and/or the sample are associated with a prognostic classification associated with the cluster.

FIG. 9 illustrates an example environment 900 for sequencing various nucleic acid molecules 902. In various implementations, the nucleic acid molecules 902 include cfDNA and/or gDNA. For instance, the nucleic acid molecules 902 may include ctDNA. The nucleic acid molecules 902, in various cases, are extracted from a sample, such as a biological sample obtained from a subject. In some implementations, the nucleic acid molecules 902 include DNA that is complementary to RNA present in the sample.

The nucleic acid molecules 902, in various cases, are ligated with adapters 904. For examples, the adapters 904 are hybridized to the nucleic acid molecules 902. The adapters 904, for example, include additional nucleic acid molecules. In various implementations, the adapters 904 have a shorter length than the nucleic acid molecules 902 being sequenced. For instance, the adapters 904 include amplification primers, flow cell adapter sequences, substrate adapter sequences, or sample index sequences. Although FIG. 9 illustrates adapters 904 being ligated to one end of each of the nucleic acid molecules 902, implementations are not so limited. For example, the adapters 904 may be ligated to both ends of each of the nucleic acid molecules 902.

In various examples, the nucleic acid molecules 902 ligated with the adapters 904 are amplified in order to generate amplified molecules 906. Various amplification techniques can be performed. For instance, the amplified molecules 906 are generated using PCR, a non-PCR amplification technique, an isothermal amplification technique, or any combination thereof.

Amplified molecules 906 may be captured by bait molecules 910 and sequenced. In some implementations, the amplified molecules 906 are sequenced via sequencing-by-synthesis. In various cases, fluorescently tagged deoxyribonucleotide triphosphates (dNTP) 912 are utilized to synthesize a strand that is complementary to DNA strands bound to the substrate 908. When a dNTP 912 is added to the strand (e.g., by an enzyme), the dNTP 912 emits an optical signal 914. In various implementations, the frequency of the optical signal 914 is dependent on the type of dNTP 912 from which the optical signal 914 is emitted. By detecting the optical signals 914 as the strand is being synthesized, the sequence of the original nucleic acid molecules 902 can be derived.

In some implementations, the amplified molecules 906 are sequenced via nanopore sequencing. For instance, the amplified molecules 906 are directed through a nanopore 916 extending through a substrate 918. In various cases, the amplified molecules 906 are negatively charged, such that they can be directed through the nanopore 916 by imposing an electrical field across the substrate 918. In various cases, the amplified molecules 906 and the nanopore 916 are in the presence of a charged solution. Thus, charged solutes traveling through the nanopore 916 can be monitored by reviewing an electrical signal (e.g., a current) sensed between electrodes 920 on either side of the substrate 918. As an amplified molecule 906 is directed through the nanopore 916, the individual bases within the amplified molecule 906 will block the nanopore 916, which may decrease the amount of charged solutes traveling through the nanopore 916 and consequently, the magnitude of the electrical signal detected by the electrodes 920. Each of the four types of bases within the amplified molecules 906, may block the nanopore 916 to a different extent. Therefore, the sequence of the nucleic acid molecules 902 can be derived by analyzing the measured electrical signal with respect to time as the amplified molecules 906 are directed through the nanopore 916.

FIG. 10 illustrates one or more devices 1000 configured to perform various operations described herein. The device(s) 1000 include one or more processor(s) 1002. In some implementations, the processor(s) 1002 includes a central processing unit (CPU), a graphics processing unit (GPU), both CPU and GPU, or other processing unit or component known in the art.

The processor(s) 1002 is operably connected to memory 1004. In various implementations, the memory 1004 is volatile (such as random access memory (RAM)), non-volatile (such as read only memory (ROM), flash memory, etc.) or some combination of the two. The memory 1004 stores instructions that, when executed by the processor(s) 1002, causes the processor(s) 1002 to perform various operations. In various examples, the memory 1004 stores methods, threads, processes, applications, objects, modules, any other sort of executable instruction, or a combination thereof. In some cases, the memory 1004 stores files, databases, or a combination thereof. In some examples, the memory 1004 includes, but is not limited to, RAM, ROM, electrically erasable programmable read-only memory (EEPROM), flash memory, or any other memory technology. In some examples, the memory 1004 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 1002. For instance, the memory 1004 stores instructions that, when executed by the processor(s) 1002, causes the processor(s) 1002 to perform operations of the feature selector 116, the predictive model 120, and the report generator 124.

The processor(s) 1002 is operably connected to one or more input devices 1006 and one or more output devices 1008. Collectively, the input device(s) 1006 and the output device(s) 1008 function as an interface between at least one user and the device(s) 1000. The input device(s) 1006 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 1008 includes at least one of a display, a speaker, a haptic output device, a printer, or any combination thereof. In various examples, the processor(s) 1002 causes a display among the input device(s) 1006 to visually output various data described herein. In some implementations, the input device(s) 1006 includes one or more touch sensors, the output device(s) 1008 includes a display screen, and the touch sensor(s) are integrated with the display screen.

In various implementations, the processor(s) 1002 is operably connected to one or more transceivers 1010 that transmit and/or receive data over one or more communication networks 1012. For example, the transceiver(s) 1010 includes a network interface card (NIC), a network adapter, a local area network (LAN) adapter, or a physical, virtual, or logical address to connect to the various external devices and/or systems. In various examples, the transceiver(s) 1010 includes any sort of wireless transceivers capable of engaging in wireless communication (e.g., radio frequency (RF) communication). For example, the communication network(s) 1012 includes one or more wireless networks that include a 3rd Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 1010 includes other wireless modems, such as a modem for engaging in WI-FI®, WIGIG®, WIMAX®, BLUETOOTH®, or infrared communication over the communication network(s) 1012.

The device(s) 1000 may further include the sequencer 112. In various implementations, the sequencer 112 includes one or more fluidic circuits 1014 configured to receive a sample 1016 derived from a subject 1018. The sequencer 112, in various cases, may be configured to generate data indicative of one or more sequences of nucleic acid molecules (e.g., DNA and/or RNA) present in the sample 1016. In various cases, the sequencer 112 introduces one or more reagents to the fluidic circuit(s) 1014 in order to prepare for and perform sequencing of the nucleic acid molecules. Further, the sequencer 112 may include one or more sensors 1020 configured to measure or otherwise detect detection signals from the fluidic circuit(s) 1014, which may be indicative of the sequences of the nucleic acid molecules. According to various implementations, the sensor(s) 1020 may further include one or more ADCs. The sequencer 112, in various cases, outputs sequence read data to the processor(s) 1002 for additional processing.

### Example Clauses

1. A method for classifying cancer, the method including: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, all or a subset of the captured amplified nucleic acid molecules to obtain a plurality of sequence reads that represent the sequenced amplified nucleic acid molecules thereby generating sequence read data representing a genome of the sample; receiving, at one or more processors, sequence read data for the plurality of sequence reads; determining, using the one or more processors, features of the sample based on the plurality of sequence reads, the features including: at least one mismatch repair deficiency (MMRD) probability score of the sample, the at least one MMRD probability score being indicative of at least one of one or more variants in at least one mismatch repair (MMR) gene, a methylation status of the at least one MMR gene, or a methylation status of one or more promotors associated with the at least one MMR gene; and a copy number state of at least one genetic loci based on the nucleic acid molecules of the sample; generating, using the one or more processors, input data indicating the features; determining, using the one or more processors, at least one cluster in a clustering model corresponding to the input data; and determining a prognostic classification of the sample based on the at least one cluster in the clustering model.

2. The method of clause 1, wherein the sample is obtained from an endometrial tumor of the subject; and/or wherein the features further include at least one of: a presence of a pathogenic variant in one or more of polymerase E (POLE), TP53, CTNNNB1, LICAM, PTEN, ERBB2, PMS2, MSH2, MSH6, MLH1, an estrogen receptor (ER) gene, or a progesterone receptor (PR) gene; a fraction unstable score;
a mutation signature; a tumor mutational burden (TMB) score; a presence of one or more hotspot mutations; a tumor purity; or a presence of one or more aneuploidy events.

3. The method of clause 1 or 2, further including: generating, using the one or more processors, a report indicating the prognostic classification; and outputting the report.

4. The method of any of clauses 1 to 3, wherein the subject has at least one of endometrial cancer, bladder cancer, kidney cancer, breast cancer.

5. The method of any of clauses 1 to 4, further including: receiving, by the one or more processors, training data including population features of a population omitting the subject; and identifying, using the one or more processors, and based on the training data, a plurality of clusters of the population features, the plurality of clusters including the at least one cluster, wherein determining the prognostic classification of the sample is performed after optimizing parameters of the clustering model.

6. The method of clause 5, wherein identifying, using the one or more processors, a plurality of clusters of the population features includes: defining, using the one or more processors, the population features into preliminary clusters in a feature space; and generating the plurality of clusters by merging, using the one or more processors, the preliminary clusters using agglomerative hierarchical clustering.

7. The method of clause 5 or 6, wherein identifying, using the one or more processors, a plurality of clusters of the population features includes: defining, using the one or more processors, the population features into at least one preliminary cluster in a feature space; and generating the plurality of clusters by splitting, using the one or more processors, the at least one preliminary cluster using divisive hierarchical clustering.

8. The method of any of clauses 5 to 7, wherein identifying, using the one or more processors, the plurality of clusters of the population features includes: defining, using the one or more processors, the population features in a multi-dimensional feature space; mapping, using the one or more processors, the population features to a two-dimensional feature space using multi-dimensional scaling; and generating the plurality of clusters by clustering, using the one or more processors, the population features in the two-dimensional feature space.

9. The method of clause 8, wherein clustering, using the one or more processors, the population features in the two-dimensional feature space includes performing, on the population features in the two-dimensional feature space, at least one of k-means clustering, density-based clustering, centroid-based clustering, spectral clustering, or distribution-based clustering.

10. The method of any of clauses 5 to 9, wherein the clustering model is a machine learning (ML) model, and the plurality of clusters of the population features is identified by training the ML model based at least in part on the training data.

11. The method of any of clauses 5 to 10, further including: correlating, by the one or more processors, individual clusters, of the plurality of clusters, with associated disease subtypes.

12. The method of any of clauses 5 to 11, further including: correlating, by the one or more processors, individual clusters, of the plurality of clusters, with associated prognostic classifications.

13. The method of clause 12, wherein determining the prognostic classification of the sample includes: determining, by the one or more processors, a particular cluster, of the plurality of clusters, that corresponds with the features indicated by the input data; and identifying, by the one or more processors, the prognostic classification associated with the particular cluster.

14. A method including: determining features of a sample from a subject, the features including one or more of: at least one MMRD probability score of the sample, the at least one MMRD probability score being indicative of at least one of one or more pathogenic variants in at least one MMR gene, a methylation status of the at least one MMR gene, or a methylation status of one or more promotors associated with the at least one MMR gene; a copy number state of at least one genetic loci based on nucleic acid molecules of the sample; a presence of a pathogenic variant in one or more of POLE, TP53, CTNNB1, LICAM, PTEN, ERBB2, PMS2, MSH2, MSH6, MLH1, an ER gene, or a PR gene; a fraction unstable score; a mutation signature; a TMB score; a tumor purity; a presence of one or more hotspot mutations; or a presence of one or more aneuploidy events; generating input data indicating the features; determining that the input data corresponds to at least one cluster in a clustering model; and determining a prognostic classification of the subject based on the at least one cluster.

15. The method of clause 14, further including: receiving a plurality of nucleic acid molecules obtained from the sample; ligating one or more adapters onto one or more nucleic acid molecules from the plurality of nucleic acid molecules; amplifying the one or more ligated nucleic acid molecules; capturing all or a subset of the amplified nucleic acid molecules; and sequencing, by a sequencer, the captured nucleic acid molecules to obtain a plurality of sequence reads that represent the captured nucleic acid molecules, thereby generating sequence read data for a genome of the sample, wherein the input data includes the sequence read data.

16. The method of clause 15, wherein the one or more adapters include amplification primers, flow cell adaptor sequences, substrate adapter sequences, or sample index sequences.

17. The method of clause 15 or 16, wherein the captured nucleic acid molecules are captured from the amplified nucleic acid molecules by hybridization to one or more bait molecules.

18. The method of clause 17, wherein the one or more bait molecules include one or more additional nucleic acid molecules, each of the one or more additional nucleic acid molecules including a region that is complementary to a region of a captured nucleic acid molecule.

19. The method of any of clauses 15 to 18, wherein amplifying the one or more ligated nucleic acid molecules includes performing a polymerase chain reaction (PCR) amplification technique, a non-PCR amplification technique, or an isothermal amplification technique.

20. The method of any of clauses 15 to 19, wherein sequencing the captured nucleic acid molecules includes use of a massively parallel sequencing (MPS) technique, whole genome sequencing (WGS), whole exome sequencing, targeted sequencing, direct sequencing, or Sanger sequencing.

21. The method of any of clauses 15 to 20, wherein sequencing the captured nucleic acid molecules includes next generation sequencing (NGS).

22. The method of any of clauses 15 to 21, wherein the sequencer includes a next generation sequencer.

23. The method of any of clauses 15 to 22, wherein sequencing the captured nucleic acid molecules includes sequencing-by-synthesis or nanopore sequencing.

24. The method of any of clauses 14 to 23, further including: generating ligated molecules by ligating adaptors onto nucleic acid molecules of the sample; generating amplified ligated molecules by amplifying the ligated molecules; generating, using the amplified ligated molecules, detection signals; detecting, by at least one sensor, the detection signals; and generating sequence read data based on the detection signals, wherein the input data includes the sequence read data.

25. The method of clause 24, wherein the detection signals include electrical signals and/or optical signals.

26. The method of clause 24 or 25, wherein generating, using the amplified ligated molecules, the detection signals includes: synthesizing, by a polymerase using fluorescently tagged nucleotide triphosphates (NTPs), a synthesized nucleic acid molecule that is complementary to one of the amplified ligated molecules, and wherein detecting, by the at least one sensor, the detection signals includes: detecting, by at least one optical sensor, optical signals emitted by the fluorescently tagged NTPs upon binding to the synthesized nucleic acid molecule, the optical signals being indicative of at least one sequence of the nucleic acid molecules of the sample.

27. The method of any of clauses 24 to 26, wherein generating, using the amplified ligated molecules, the detection signals includes: directing the amplified ligated molecules through a nanopore extending from a first space to a second space through a substrate, and wherein detecting, by the at least one sensor, the detection signals includes: detecting, by sensors disposed in the first space and the second space, an electrical signal over time, the electrical signal being indicative of at least one sequence of the nucleic acid molecules of the sample.

28. The method of any of clauses 24 to 27, wherein the sequence read data indicates a full genome or RNA transcriptome of the sample.

29. The method of any of clauses 24 to 28, wherein the sequence read data indicates a whole exome of the sample.

30. The method of any of clauses 24 to 29, wherein the sequence read data indicates a predetermined panel of genes of the sample.

31. The method of clause 30, wherein the predetermined panel includes MSH2, MSH6, PMS2, and MLH1.

32. The method of clause 30 or 31, wherein the predetermined panel includes one or more of TP53, PTEN, POLE, MKI67, FAT3, TAF1, ZFHX3, RPL22, SPTA1, FAM135B, CSMD3, GIGYF2, CSDE1, MLL4, ATR, CTNNB1, USH2A, LIMCH1, RRN3P2, FBXW7, CDH19, USP9X, COL11A1, BCOR, ARIDIA, ZNF770, ARID5B, SLC9A11, KRAS, PNN, INPP4A, CTCF, CHD4, AMY2B, RBMX, PPP2RIA, TNFAIP6, PIK3R1, SGK1, HOXA7, METTL14, HPD, MIR1277, CCND1, MECOM, NFE2L2, or ESR1.

33. The method of any of clauses 30 to 32, wherein the predetermined panel includes one or more of ABL1, ACVR1B, AKT1, AKT2, AKT3, ALK. ALOX12B, AMER1, APC, AR, ARAF, ARFRP1, ARIDIA, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1. FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNFIA, HRAS, HSD3B1, ID3, IDH1, IDH2, IGFIR, IKBKE, IKZF1, INPP4B, IRF2, IRF4, IRS2, JAK1, JAK2, JAK3, JUN, KDM5A, KDM5C, KDM6A, KDR. KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP. MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKARIA, PRKCI, PTCH1, PTEN, PTPN11, PTPRO, QKI, RAC1, RAD21, RAD51, RAD51B, RAD51C. RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC. STAG2, STAT3, STK11, SUFU, SYK, TBX3, TEK, TERC, TERT, TET2, TGFBR2, TIPARP. TMPRSS2, TNFAIP3, TNFRSF14, TP53, TSC1, TSC2, TYRO3, U2AF1, VEGFA, VHL, WHSC1, WHSCIL1. WT1, XPO1, XRCC2, ZNF217, ZNF703, ABL, ALK, ALL, B4GALNT1, BAFF, BCL2, BRAF. BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1B, IL-6, IL-6R, JAK1, JAK2, JAK3, KIT, KRAS, MEK, MET, MSI-H, mTOR, PARP. PD-1, PDGFR, PDGFRa, PDGFRB, PD-L1, PI3K8, PIGF, PTCH, RAF, RANKL, RET. ROS1, SLAMF7, VEGF, VEGFA, or VEGFB.

34. The method of any of clauses 24 to 33, further including: receiving the sample.

35. The method of clause 34, wherein the sample includes a tissue biopsy sample, a liquid biopsy sample, or a normal control.

36. The method of clause 34 or 35, wherein the sample is a liquid biopsy sample and includes blood, plasma, cerebrospinal fluid, sputum, stool, urine, lymphatic fluid, or saliva.

37. The method of any of clauses 34 to 36, wherein the sample is a liquid biopsy sample and includes circulating tumor cells (CTCs).

38. The method of any of clauses 34 to 37, wherein the sample is a liquid biopsy sample and includes cell-free DNA (cfDNA), circulating tumor DNA (ctDNA), or any combination thereof.

39. The method of any of clauses 34 to 38, further including extracting DNA or RNA from the sample.

40. The method of clause 39, wherein the DNA includes genomic DNA or cDNA.

41. The method of clause 39 or 40, wherein the RNA includes messenger RNA, microRNA, or non-coding RNA.

42. The method of any of clauses 24 to 41, wherein determining the features of the sample includes: determining, based on the sequence read data, a mutational profile of the sample; inputting the mutational profile into a model, wherein the model is trained using training data related to a plurality of mutational signatures; and predicting one or more mutational signatures of the plurality of mutational signatures associated with the sample based on an output of the model, wherein the output of the model is associated with a dimensionality value that is less than a number of the plurality of mutational signatures, and wherein the features include the one or more mutational signatures.

43. The method of clause 42, wherein the model includes an autoencoder model.

44. The method of any of clauses 24 to 43, wherein determining the features of the sample includes: determining, based on the sequence read data, the MMRD probability score being indicative of a functional deficiency in at least one mismatch repair gene, wherein the features include the MMRD probability score.

45. The method of clause 44, the features being first features, the input data being first input data, wherein determining, based on the sequence read data, the MMRD probability score includes: generating, by extracting two or more second features of the sequence read data, second input data; and inputting the second input data into a predictive model configured to generate the MMRD probability score based on the second input data.

46. The method of any of clauses 24 to 45, wherein determining the features of the sample includes: determining, based on the sequence read data, the copy number state, and wherein the features include the copy number state.

47. The method of clause 46, wherein determining, based on the sequence read data, the copy number state includes: generating, based on the sequence read data, a major allele coverage ratio and a minor allele coverage ratio; segmenting one or more nucleic acid sequences associated with the sequence read data into segments; generating copy number grid model input data including: a sum of the major allele coverage ratio and the minor allele coverage ratio; and a difference of the major allele coverage ratio and the minor allele coverage ratio; fitting copy number grid models including allowed copy number states to the copy number grid model input data; selecting a copy number grid model among the copy number grid models; and assigning the copy number state for at least a portion of the one or more nucleic acid sequences based on the selected copy number grid model.

48. The method of any of clauses 24 to 47, further including determining, based on the sequence read data, an MSI fraction, the fraction unstable score including the MSI fraction.

49. The method of any of clauses 14 to 48, wherein the prognostic classification is selected from POLE ultra-mutated, microsatellite instability hypermutated, copy number low, or copy number high, and/or wherein the prognostic classification is selected from: a first classification associated with pathogenic mutations in POLE; a second classification associated with mismatch repair deficiency; a third classification associated with mutations in TP53; or a fourth classification associated with an absence of the pathogenic mutations in POLE, an absence of mismatch repair deficiency, and an absence of the mutations in TP53.

50. The method of any of clauses 14 to 49, wherein the features include a presence of at least one pathogenic variant in one or more of POLE, MLH1, PMS2, MSH2, MSH6, or TP53.

51. The method of any of clauses 14 to 50, wherein the features include a number of pathogenic variants in one or more of POLE, MLH1, PMS2, MSH2, MSH6, or TP53.

52. The method of any of clauses 14 to 51, wherein the features include pathogenic variants in at least one of POLE, TP53, CTNNNB1, LICAM, PTEN, ERBB2, PMS2, MSH2, MSH6, MLH1, the ER gene, or the PR gene.

53. The method of any of clauses 14 to 52, wherein the subject has at least one of endometrial cancer, bladder cancer, kidney cancer, or breast cancer.

54. The method of any of clauses 14 to 53, wherein the sample is obtained from a tumor of the subject.

55. The method of any of clauses 14 to 54, wherein determining that the input data corresponds to the at least one cluster in the clustering model includes: determining a distance between the input data and the at least one cluster in a feature space; and determining that the distance is less than a threshold distance.

56. The method of clause 55, wherein the distance includes at least one of a Euclidian distance, a Manhattan distance, a Hamming distance, a Minkowski distance, or a Chebyshev distance.

57. The method of clause 55 or 56, wherein the distance is between the input data and: at least one population feature in the feature space; a border of the at least one cluster; or a centroid of the at least one cluster.

58. The method of any of clauses 14 to 57, wherein determining that the input data corresponds to the at least one cluster in the clustering model includes: determining that the input data is within the at least cluster in a feature space.

59. The method of any of clauses 14 to 58, wherein the clustering model includes at least one of a hierarchical clustering model, a k-means clustering model, a density-based clustering model, a spectral clustering model, a centroid-based clustering model, or a distribution-based clustering model.

60. The method of any of clauses 14 to 59, further including: identifying training data including population features of population samples obtained from a population omitting the subject; and generating the clustering model by clustering the training data.

61. The method of clause 60, wherein generating the clustering model by clustering the training data includes: defining the population features into preliminary clusters in a feature space; and generating a plurality of clusters by merging the preliminary clusters using agglomerative hierarchical clustering, the plurality of clusters including the at least one cluster.

62. The method of clause 61, wherein merging the preliminary clusters using agglomerative hierarchical clustering includes: determining a distance between a first preliminary cluster among the preliminary clusters and a second preliminary cluster among the preliminary clusters; determining that the distance is below a threshold distance; and merging the first preliminary cluster and the second preliminary cluster.

63. The method of clause 62, wherein determining the distance between the first preliminary cluster and the second preliminary cluster includes determining at least one of: a mean distance between the population features in the first preliminary cluster and the population features in the second preliminary cluster; a maximum distance between the population features in the first preliminary cluster and the population features in the second preliminary cluster; or a minimum distance between the population features in the first preliminary cluster and the population features in the second preliminary cluster.

64. The method of any of clauses 60 to 63, wherein generating the clustering model by clustering the training data includes: defining the population features into at least one preliminary cluster in a feature space; and generating a plurality of clusters by splitting the at least one preliminary cluster using divisive hierarchical clustering, the plurality of clusters including the at least one cluster.

65. The method of any of clauses 60 to 64, wherein generating the clustering model by clustering the training data includes: defining the population features in an n-dimensional feature space; mapping the population features to an m-dimensional feature space using multi-dimensional scaling, m being less than n; and generating at least one cluster by clustering the population features in the m-dimensional feature space.

66. The method of clause 65, wherein clustering the population features in the m-dimensional feature space includes performing, on the population features in the m-dimensional feature space, at least one of k-means clustering, density-based clustering, centroid-based clustering, spectral clustering, or distribution-based clustering.

67. The method of any of clauses 60 to 66, wherein the population features further include prognostic classifications of the population.

68. The method of any of clauses 60 to 67, wherein the clustering model is a machine learning (ML) model, and generating the clustering model includes: training the ML model, based on the training data, by identifying a plurality of clusters of the population features.

69. The method of clause 68, further including: correlating individual clusters, of the plurality of clusters, with associated disease subtypes.

70. The method of clause 68 or 69, further including: correlating individual clusters, of the plurality of clusters, with associated prognostic classifications.

71. The method of clause 70, wherein determining the prognostic classification of the subject includes: identifying the at least one cluster, of the plurality of clusters, that corresponds with the features indicated by the input data; and identifying the prognostic classification associated with the at least one cluster.

72. The method of any of clauses 14 to 71, wherein the prognostic classification includes a disease subtype.

73. The method of any of clauses 14 to 72, wherein the prognostic classification includes at least one of: a predicted pathology of the subject; a predicted pathology subtype of the subject; a predicted survivability of the subject; a predicted symptom of the subject; a predicted effective therapy to treat the predicted pathology of the subject; a predicted stage of the predicted pathology of the subject; a predicted grade of the predicted pathology of the subject; a predicted Eastern Cooperative Oncology Group (ECOG) performance status of the subject; a predicted activity level of the subject; a predicted diabetes status of the subject; a predicted body mass index (BMI) of the subject; a predicted smoking history of the subject; a predicted breast density of the subject; a clinical trial that the subject is predicted to qualify for; or a characteristic of the predicted pathology of the subject.

74. The method of clause 73, wherein the predicted pathology includes at least one cancer type and/or cancer subtype.

75. The method of any of clauses 14 to 74, further including: generating, based on the prognostic classification, a genomic profile of a subject from whom the sample was obtained.

76. The method of clause 75, wherein the genomic profile includes results from at least one of: a comprehensive genomic profiling test; a gene expression profiling test; a cancer hotspot panel test; a DNA methylation test; a DNA fragmentation test; or an RNA fragmentation.

77. The method of clause 75 or 76, wherein the genomic profile of the subject includes: results from a nucleic acid sequencing-based test.

78. The method of any of clauses 75 to 77, further including: selecting, based on the genomic profile and/or the prognostic classification, an anticancer agent for administration to the subject.

79. The method of clause 78, further including: administering the anticancer agent to the subject.

80. The method of any of clauses 75 to 79, further including: applying, based on the genomic profile, an anticancer therapy to the subject based on the generated genomic profile.

81. The method of clause 80, wherein the anticancer therapy includes at least one of chemotherapy, radiation therapy, immunotherapy, a targeted therapy, or surgery.

82. The method of any of clauses 14 to 81, further including: identifying, based on the prognostic classification, a suggested treatment decision for a subject associated with the sample.

83. The method of clause 82, wherein the suggested treatment decision includes radiotherapy and/or chemotherapy.

84. The method of any of clauses 14 to 83, further including: generating a report indicating the prognostic classification; and outputting the report.

85. The method of clause 84, wherein outputting the report includes: transmitting data indicating the report to an external device.

86. The method of clause 85, wherein the external device is associated with a subject associated with the sample or a healthcare provider.

87. The method of clause 85 or 86, wherein the data is transmitted over one or more communication networks.

88. The method of any of clauses 85 to 87, wherein the data is transmitted over a peer-to-peer connection.

89. The method of any of clauses 84 to 88, wherein outputting the report includes: visually presenting, by a display, the report.

90. The method of any of clauses 84 to 89, further including: determining, based on the prognostic classification, one or more therapies to treat a pathology of the subject, wherein the report further indicates the one or more therapies.

91. The method of clause 90, wherein the pathology includes at least one of endometrial cancer, bladder cancer, kidney cancer, or breast cancer.

92. The method of any of clauses 14 to 91, further including: generating, based on the prognostic classification, a therapy for the subject.

93. The method of clause 92, wherein the therapy includes a dosage of one or more therapeutic agents predicted to treat a pathology of the subject.

94. The method of any of clauses 14 to 93, further including: determining, based on the prognostic classification, whether a subject associated with the sample is eligible for a clinical trial.

95. A method, including: identifying training data including features of samples of a population of individuals, the features including one or more of: MMRD probability scores of the samples; copy number states of one or more genetic loci based on nucleic acid molecules of the samples; generating input data indicating the features; pathogenic variants in one or more of POLE, TP53, CTNNB1, LICAM, PTEN, ERBB2, PMS2, MSH2, MSH6, MLH1, an ER gene, or a PR gene of the samples; fraction unstable scores of the samples; mutation signatures of the samples; TMB scores of the samples; presences of one or more hotspot mutations; tumor purity; or presences of one or more aneuploidy events; generating a clustering model by clustering the training data into a plurality of clusters; and assigning prognostic classifications to the plurality of clusters based on prognostic outcomes of the population.

96. The method of clause 95, wherein the training data includes sequence read data of the samples, the sequence read data indicating at least one of full genomes of the samples, RNA transcriptomes of the samples, whole exomes of the samples, or a predetermined panel of genes of the samples.

97. The method of clause 96, wherein the predetermined panel includes MSH2, MSH6, PMS2, and MLH1.

98. The method of clause 96 or 97, wherein the predetermined panel includes one or more of TP53, PTEN, ERBB2, PMS2, MSH2, MSH6, MLH1, POLE, MKI67, FAT3, TAF1, ZFHX3, RPL22, SPTA1, FAM135B, CSMD3, GIGYF2, CSDE1, MLL4, ATR, CTNNB1, USH2A, LIMCH1, RRN3P2, FBXW7, CDH19, USP9X, COL11A1, BCOR, ARIDIA, ZNF770, ARID5B, SLC9A11, KRAS, PNN, INPP4A, CTCF, CHD4, AMY2B, RBMX, PPP2RIA, TNFAIP6, PIK3R1, SGK1, HOXA7, METTL14, HPD, MIR1277, CCND1, MECOM, NFE2L2, or ESR1.

99. The method of any of clauses 95 to 98, wherein the training data includes features based on histological images of the samples.

100. The method of any of clauses 95 to 99, wherein generating the clustering model by clustering the training data includes: defining the features into preliminary clusters in a feature space; and generating the plurality of clusters by merging the preliminary clusters using agglomerative hierarchical clustering.

101. The method of clause 100, wherein merging the preliminary clusters using agglomerative hierarchical clustering includes: determining a distance between a first preliminary cluster among the preliminary clusters and a second preliminary cluster among the preliminary clusters; determining that the distance is below a threshold distance; and merging the first preliminary cluster and the second preliminary cluster.

102. The method of clause 101, wherein determining the distance between the first preliminary cluster and the second preliminary cluster includes determining at least one of: a mean distance between the features in the first preliminary cluster and the features in the second preliminary cluster; a maximum distance between the features in the first preliminary cluster and the features in the second preliminary cluster; or a minimum distance between the features in the first preliminary cluster and the features in the second preliminary cluster.

103. The method of any of clauses 100 to 102, wherein generating the clustering model by clustering the training data includes: defining the features into at least one preliminary cluster in a feature space; and generating the plurality of clusters by splitting the at least one preliminary cluster using divisive hierarchical clustering.

104. The method of any of clauses 95 to 103, wherein generating the clustering model by clustering the training data includes: defining the features in an n-dimensional feature space; mapping the features to an m-dimensional feature space using multi-dimensional scaling, m being less than n; and generating the plurality of clusters by clustering the features in the m-dimensional feature space.

105. The method of clause 104, wherein clustering the features in the m-dimensional feature space includes performing, on the features in the m-dimensional feature space, at least one of k-means clustering, density-based clustering, centroid-based clustering, spectral clustering, or distribution-based clustering.

106. The method of any of clauses 95 to 105, wherein assigning prognostic classifications to the plurality of clusters based on prognostic outcomes of the population includes: identifying a portion of the individuals corresponding to a cluster among the plurality of clusters; determining that greater than a threshold amount of the portion of the individuals are associated with a common prognostic outcome; and assigning the common prognostic outcome to the cluster.

107. The method of any of clauses 95 to 106, wherein assigning prognostic classifications to the plurality of clusters based on prognostic outcomes of the population includes: identifying a portion of the individuals corresponding to a cluster among the plurality of clusters; determining an average prognostic outcome based on the prognostic outcomes of the portion of the individuals; and assigning the average prognostic outcome to the cluster.

108. A system, including: at least one processor; and memory storing instructions that, when executed by the at least one processor, cause the at least one processor to perform operations including: the method of any of clauses 14 to 107.

109. A non-transitory computer-readable medium storing instructions for performing operations including: the method of any of clauses 14 to 107.

### CONCLUSION

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on.” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at Icast be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range, to within a range of +20% of the stated value; +19% of the stated value; +18% of the stated value; +17% of the stated value; +16% of the stated value; +15% of the stated value; +14% of the stated value; +13% of the stated value; +12% of the stated value; +11% of the stated value; +10% of the stated value; +9% of the stated value; +8% of the stated value; +7% of the stated value; +6% of the stated value; +5% of the stated value; +4% of the stated value; +3% of the stated value; +2% of the stated value; or +1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

Tumor mutational burden (TMB) is a measure of the number of mutations carried by tumor cells. By comparing DNA sequences from a patient's healthy tissues and tumor cells, the number of acquired somatic mutations present in tumors, but not in normal tissues, may be determined. In some instances, driver mutations may be excluded from a TMB calculation.

In certain examples, “tumor mutational burden” or “TMB” refers to the number of somatic mutations in a tumor's genome and/or the number of somatic mutations per area of the tumor's genome. In some embodiments, TMB, as used herein, refers to the number of somatic mutations per megabase (Mb) of DNA sequenced. In some embodiments, germline (inherited) variants are excluded when determining TMB, given that the immune system has a higher likelihood of recognizing these as self. In various cases, driver mutations are excluded from a TMB calculation.

Microsatellites are highly polymorphic DNA-repeat regions. In certain examples. “microsatellite” refers to a repetitive nucleic acid having repeat units of less than about 10 base pairs or nucleotides in length. In certain examples, a microsatellite refers to a tract of tandemly repeated (i.e. adjacent) DNA motifs ranging from one to six or up to ten nucleotides, with each motif repeated 5 to 50 repeated times. “Microsatellite instability” refers to genetic instability in the microsatellite regions. Cancer patients with microsatellite instability classified as being high (MSI-H or MSI-High) frequently exhibit an accumulation of somatic mutations in tumor cells that leads to a range of molecular and biological changes including high tumor mutational burden, increased expression of neoantigens and abundant tumor-infiltrating lymphocytes. Chang et al. “Microsatellite Instability: A Predictive Biomarker for Cancer Immunotherapy.” Appl Immunohistochem Mol Morphol. 26(2):e15-e21 (2018). These changes have been linked to increased sensitivity to checkpoint inhibitor drugs, such as pembrolizumab, which is used to treat advanced melanoma, head and neck squamous cell carcinoma, non-small cell lung cancer (NSCLC), and classical Hodgkin lymphoma.

A viral status test refers to a test that identifies the presence of viral RNA or DNA in a subject. The test can identify viral load and/or viral identity. For example, the viral status test can identify the presence of viral RNA or DNA associated with the occurrence of certain cancers. Examples of such viruses include Hepatitis B Virus (HBV) and Hepatitis C Virus (HCV), Kaposi Sarcoma-Associated Herpesvirus (KSHV), Merkel Cell Polyomavirus (MCV), Human Papillomavirus (HPV). Human Immunodeficiency Virus Type 1 (HIV-1, or HIV), Human T-Cell Lymphotropic Virus Type 1 (HTLV-1), and Epstein-Barr Virus (EBV).

Cancer “hotspot” mutations give rise to oncological outcomes. PhyloP. SIFT, Grantham, COSMIC and PolyPhen-2 are in silico tools that can be used to assess pathogenicity of identified variants. Exemplary hotspot genes and mutations include EGFR exon 19 activating mutation, EGFR exon 19 deletion, EGFR exon 19 insertion, EGFR exon 19 sensitizing mutation, EGFR exon 20 activation mutation, EGFR exon 20 insertion, EGFR G719 mutation, EGFR L858R mutation, EGFR L861 mutation, EGFR S768 mutation, EGFR T790M mutation, C797 mutation, KIT activating mutation, KRAS activating mutation, MET activating mutation, NRAS activating mutation, PMS2 promoter mutations, among many others. Hotspot mutations also occur in the following genes: AKT2, BRCA1, BRCA2, ERC1, NSD1, POLH, PPMIG, PTEN, RAD18, RAD51, RAD51B, RB1, TERT, TP53, TP53Bp1, ALK, ARMTI, ATAD5, ATG7, ATIC, AXL, BIRC6, BRD3, BRD4, CAPRIN1, CCAR2, CCDC6, CDK5RAP2, CHD9, CIT, CTNNB1, CUL1, EBF1, EIF3E, HIP1, HMGA2, IRF2BP2, NOTCH1, NOTCH4, NPM1, OFD1, TACC1, TACC3, TERF2, TMEM106B, UBE2L3, USP10, WRDR48, YAP1, ZEB2, and ZMYND8.

A “DNA methylation test” refers to an assay, which can be commercially available, for distinguishing methylated versus unmethylated cytosine loci in DNA. Techniques for measuring cytosine methylation include bisulfite-based methylation assays. The addition of bisulfite to DNA results in the methylation of unmethylated cytosine and its ultimate conversion to the nucleotide uracil. Uracil has similar binding properties to thiamine in the DNA sequence. Previously methylated cytosine does not undergo similar chemical conversion on exposure to bisulfite. Bisulfite assays can thus be used to discriminate previously methylated versus unmethylated cytosine.

An exemplary quantitative methylation detection assay combines bisulfite treatment and restriction analysis COBRA, which uses methylation sensitive restriction endonucleases, gel electrophoresis, and detection based on labeled hybridization probes. (Ziong and Laird, Nucleic Acid Res. 1997 25; 2532-4). Another exemplary detection assay is the methylation specific polymerase chain reaction PCR (MSPCR) for amplification of DNA segments of interest. This assay can be performed after sodium bisulfite conversion of cytosine and uses methylation sensitive probes. Other detection assays include the Quantitative Methylation (QM) assay, which combines PCR amplification with fluorescent probes designed to bind to putative methylation sites; MethyLight™ (Qiagen, Redwood City, CA) a quantitative methylation detection assay that uses fluorescence-based PCR (Eads, et al., Cancer Res. 1999; 59:2302-2306); and Ms-SNuPE, a quantitative technique for determining differences in methylation levels in CpG sites. As with other techniques, Ms-SNuPE also requires bisulfite treatment to be performed first, leading to the conversion of unmethylated cytosine to uracil while methyl cytosine is unaffected. PCR primers specific for bisulfite converted DNA are then used to amplify the target sequence of interest. The amplified PCR product is isolated and used to quantitate the methylation status of the CpG site of interest. (Gonzalgo and Jones Nuclei Acids Res 1997; 25:252-31).

In particular embodiments, pyrosequencing can be used to detect marker methylation. Pyrosequencing is a method of DNA sequencing that relies on detection of the release of pyrophosphates as DNA is synthesized (and is therefore a “sequencing by synthesis” technique). To assess methylation by pyrosequencing, a DNA sample can be incubated with sodium bisulfite, converting unmethylated cytosine to uracil. The presence of uracil will result in thymine incorporation during PCR amplification. Therefore, sequencing results that include thymine at a nucleotide position that is known to encode cytosine can be interpreted as unmethylated sites. In contrast cytosines present in the sequencing results indicate that the site was methylated in the original DNA sample, because methylation protects cytosine from conversion to uracil upon treatment. Bisulfite treatment can also be performed on control samples with known methylation patterns, to reduce or eliminate false positive results. Commercially available pyrosequencing machines include Pyro Mark Q96 (Qiagen, Hilden, Germany). For more details on methods to use pyrosequencing for measurement of methylation, see Delaney et al. Methods Mol Biol. 2015 1343:249-264. Pyrosequencing is especially useful for detecting methylation in the CpG sites within genes.

In particular embodiments, a protein marker is detected by contacting a sample with reagents (e.g., antibodies), generating complexes of reagent and marker(s), and detecting the complexes. Particular embodiments for detecting and measuring protein levels can use methods including agglutination, chemiluminescence, electro-chemiluminescence (ECL), enzyme-linked immunoassays (ELISA), immunoassay, immunoblotting, immunodiffusion, immunoelectrophoresis, immunofluorescence, immunohistochemistry, immunoprecipitation, mass-spectrometry, and western blot. See also, e.g., E. Maggio, Enzyme-Immunoassay (1980), CRC Press, Inc., Boca Raton, Fla; and U.S. Pat. Nos. 4,727,022; 4,659,678; 4,376,110; 4,275,149; 4,233,402; and 4,230,797.

Read depth refers to the number of times that a specific genomic site is sequenced during a sequencing run.

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A method for classifying cancer, the method comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, all or a subset of the captured amplified nucleic acid molecules to obtain a plurality of sequence reads that represent the sequenced amplified nucleic acid molecules thereby generating sequence read data representing a genome of the sample;
receiving, at one or more processors, sequence read data for the plurality of sequence reads;
determining, using the one or more processors, features of the sample based on the plurality of sequence reads, the features comprising:
at least one mismatch repair deficiency (MMRD) probability score of the sample, the at least one MMRD probability score being indicative of at least one of one or more variants in at least one mismatch repair (MMR) gene, a methylation status of the at least one MMR gene, or a methylation status of one or more promotors associated with the at least one MMR gene; and
a copy number state of at least one genetic loci based on the nucleic acid molecules of the sample;
generating, using the one or more processors, input data indicating the features;
determining, using the one or more processors, at least one cluster in a clustering model corresponding to the input data; and
determining a prognostic classification of the sample based on the at least one cluster in the clustering model.

2. The method of claim 1, wherein the sample is obtained from an endometrial tumor of the subject; and/or
wherein the features further comprise at least one of:
a presence of a pathogenic variant in one or more of polymerase E (POLE), TP53, CTNNNB1, LICAM, PTEN, ERBB2, PMS2, MSH2, MSH6, MLH1, an estrogen receptor (ER) gene, or a progesterone receptor (PR) gene;
a fraction unstable score;
a mutation signature;
a tumor mutational burden (TMB) score;
a presence of one or more hotspot mutations;
a tumor purity; or
a presence of one or more aneuploidy events.

3. The method of claim 1, further comprising:
generating, using the one or more processors, a report indicating the prognostic classification; and
outputting the report.

4. The method of claim 1, wherein the subject has at least one of endometrial cancer, bladder cancer, kidney cancer, breast cancer.

5. The method of claim 1, further comprising:
receiving, by the one or more processors, training data comprising population features of a population omitting the subject; and
identifying, using the one or more processors, and based on the training data, a plurality of clusters of the population features, the plurality of clusters comprising the at least one cluster,
wherein determining the prognostic classification of the sample is performed after optimizing parameters of the clustering model.

6. The method of claim 5, wherein identifying, using the one or more processors, a plurality of clusters of the population features comprises:
defining, using the one or more processors, the population features into preliminary clusters in a feature space; and
generating the plurality of clusters by merging, using the one or more processors, the preliminary clusters using agglomerative hierarchical clustering.

7. The method of claim 5, wherein identifying, using the one or more processors, a plurality of clusters of the population features comprises:
defining, using the one or more processors, the population features into at least one preliminary cluster in a feature space; and
generating the plurality of clusters by splitting, using the one or more processors, the at least one preliminary cluster using divisive hierarchical clustering.

8. The method of claim 5, wherein identifying, using the one or more processors, the plurality of clusters of the population features comprises:
defining, using the one or more processors, the population features in a multi-dimensional feature space;
mapping, using the one or more processors, the population features to a two-dimensional feature space using multi-dimensional scaling; and
generating the plurality of clusters by clustering, using the one or more processors, the population features in the two-dimensional feature space.

9. The method of claim 8, wherein clustering, using the one or more processors, the population features in the two-dimensional feature space comprises performing, on the population features in the two-dimensional feature space, at least one of k-means clustering, density-based clustering, centroid-based clustering, spectral clustering, or distribution-based clustering.

10. The method of claim 5, wherein the clustering model is a machine learning (ML) model, and the plurality of clusters of the population features is identified by training the ML model based at least in part on the training data.

11. The method of claim 5, further comprising:
correlating, by the one or more processors, individual clusters, of the plurality of clusters, with associated disease subtypes.

12. The method of claim 5, further comprising:
correlating, by the one or more processors, individual clusters, of the plurality of clusters, with associated prognostic classifications.

13. The method of claim 12, wherein determining the prognostic classification of the sample comprises:
determining, by the one or more processors, a particular cluster, of the plurality of clusters, that corresponds with the features indicated by the input data; and
identifying, by the one or more processors, the prognostic classification associated with the particular cluster.

14. A method comprising:
determining features of a sample from a subject, the features comprising one or more of:
at least one MMRD probability score of the sample, the at least one MMRD probability score being indicative of at least one of one or more pathogenic variants in at least one MMR gene, a methylation status of the at least one MMR gene, or a methylation status of one or more promotors associated with the at least one MMR gene;
a copy number state of at least one genetic loci based on nucleic acid molecules of the sample;
a presence of a pathogenic variant in one or more of POLE, TP53, CTNNB1, LICAM, PTEN, ERBB2, PMS2, MSH2, MSH6, MLH1, an ER gene, or a PR gene;
a fraction unstable score;
a mutation signature;
a TMB score;
a tumor purity;
a presence of one or more hotspot mutations; or
a presence of one or more aneuploidy events;
generating input data indicating the features;
determining that the input data corresponds to at least one cluster in a clustering model; and
determining a prognostic classification of the subject based on the at least one cluster.

15. The method of claim 14, further comprising:
receiving a plurality of nucleic acid molecules obtained from the sample;
ligating one or more adapters onto one or more nucleic acid molecules from the plurality of nucleic acid molecules;
amplifying the one or more ligated nucleic acid molecules;
capturing all or a subset of the amplified nucleic acid molecules; and
sequencing, by a sequencer, the captured nucleic acid molecules to obtain a plurality of sequence reads that represent the captured nucleic acid molecules, thereby generating sequence read data for a genome of the sample,
wherein the input data comprises the sequence read data.

16. The method of claim 15, wherein the one or more adapters comprise amplification primers, flow cell adaptor sequences, substrate adapter sequences, or sample index sequences.

17. The method of claim 15, wherein the captured nucleic acid molecules are captured from the amplified nucleic acid molecules by hybridization to one or more bait molecules.

18. The method of claim 17, wherein the one or more bait molecules comprise one or more additional nucleic acid molecules, each of the one or more additional nucleic acid molecules comprising a region that is complementary to a region of a captured nucleic acid molecule.

19. The method of claim 15, wherein amplifying the one or more ligated nucleic acid molecules comprises performing a polymerase chain reaction (PCR) amplification technique, a non-PCR amplification technique, or an isothermal amplification technique.

20. The method of claim 15, wherein sequencing the captured nucleic acid molecules comprises use of a massively parallel sequencing (MPS) technique, whole genome sequencing (WGS), whole exome sequencing, targeted sequencing, direct sequencing, or Sanger sequencing.
